SEQUENCE LISTING (11 sequences)

SEQ ID NO: 1 - protein, 4 aa, Artificial sequence
  Terminal oligopeptide for PBAE polymer
CRRR

SEQ ID NO: 2 - protein, 4 aa, Artificial sequence
  Terminal oligopeptide for PBAE polymer
CHHH

SEQ ID NO: 3 - protein, 4 aa, Artificial sequence
  Terminal oligopeptide for PBAE polymer
CKKK

SEQ ID NO: 4 - protein, 4 aa, Artificial sequence
  Terminal oligopeptide for PBAE polymer
CEEE

SEQ ID NO: 5 - protein, 4 aa, Artificial sequence
  Terminal oligopeptide for PBAE polymer
CDDD

SEQ ID NO: 6 - DNA, 27 bp, Artificial sequence
  LTR probe for integrated lentiviral vector detection
aaccattaggagtagcacccaccaagg

SEQ ID NO: 7 - DNA, 20 bp, Artificial sequence
  forward primer for LTR amplification
tggaggaggagatatgaggg

SEQ ID NO: 8 - DNA, 20 bp, Artificial sequence
  reverse primer for LTR amplification
ctgctgcactataccagaca

SEQ ID NO: 9 - DNA, 20 bp, Artificial sequence
  mouse GAPDH probe
cgcctggtcaccagggctgc

SEQ ID NO: 10 - DNA, 20 bp, Artificial sequence
  forward primer for GAPDH amplification
aacggatttggccgtattgg

SEQ ID NO: 11 - DNA, 20 bp, Artificial sequence
  reverse primer for GAPDH amplification
cattctcggccttgactgtg

(19) United States
(12) Patent Application Publication
(10) Pub. No.: US 2022/0403416 A1
(43) Pub. Date: Dec. 22, 2022

(54) Polymer-Encapsulated Viral Vectors for In Vivo Genetic Therapy

(71) Applicant: Ixaka France, Villejuif (FR)

(72) Inventors: Frédéric MOURLANE, Nice (FR); Emeline SARRY, Arcueil (FR); Renaud VAILLANT, Gentilly (FR); Cécile BAUCHE, Paris (FR); Phiilippe BISHOP, Las Vegas, NV (US); Rachel PACHERIE, Sceaux (FR)

(21) Appl. No.: 17/777,106

(22) PCT Filed: Nov. 16, 2020
(86) PCT No.: PCT/IB2020/000955

§ 371 Date: May 16, 2022

Related U.S. Application Data
(60) Provisional application No. 62/936,375, filed on Nov. 15, 2019.

(51) Int. Cl. C12N 15/86; A61K 47/69; A61K 9/51; C12N 15/88

(52) U.S. Cl. CPC C12N 15/86; A61K 47/6935; A61K 9/51; C12N 15/88; C12N 2740/16045; C12N 2740/16043

(57) ABSTRACT

Polymer-encapsulated viral vector nanoparticles and methods of using them provide enhanced delivery of genetic material for use in gene therapy and other applications. The nanoparticles include an outer shell containing an oligopeptide-modified poly(beta-amino ester) polymer which encapsulates the vector and allows the vector to transduce cells without the need for pseudotyping or the inclusion of any viral fusion protein, such as VSV-G. The polymer-encapsulated vector nanoparticles have a natural tropism for peripheral blood cells, such as leucocytes, without the need for a targeting moiety, and have an improved safety profile compared to pseudotyped viral vectors.

### CROSS-REFERENCE TO RELATED APPLICATIONS

This application claims priority to U.S. Provisional Application No. 62/936,375, filed 15 Nov. 2019, which is incorporated by reference herein in its entirety.

### BACKGROUND

Gene therapy delivers exogenous genetic material to the target cells in order to correct genetic abnormalities or provide treatment of a disease by altering cell function. To this end, both viral and non-viral gene delivery methods have been used, but both approaches still present significant shortcomings.

A number of viral vector applications have already been translated into the clinic, either for gene therapy or vaccination protocols. Nevertheless, currently used viral vectors have certain disadvantages. For example, it is challenging to target specific cells using a viral vector. In some gene therapy protocols, cell targeting is achieved by purifying the target cells and transducing them ex vivo and expanding the transduced cells in vitro before re-implanting them into the patient. In vaccination protocols, direct injection of the vector is performed, and non-specific cell transduction is often used to elicit an immune response against the gene product encoded by the vector. To increase efficacy and safety of the treatment, cell targeting could be required. Pseudotyped viral vectors can be immunogenic and an immune response developed after a first injection renders it unsafe to use repeat injections. Such viral vectors also can be difficult to target precisely and may accumulate in undesired organs and tissues.

There is a need for improved viral vector-based systems for targeted delivery of genetic material.

### SUMMARY

The technology described herein provides polymer-encapsulated viral vector nanoparticles and methods of using them to provide enhanced delivery of genetic material for use in gene therapy and other applications. The vectors and methods can be employed in any situation for which transduction of cells with one or more transgenes is useful. For example, they can be used for treatment of cancer, infectious diseases, metabolic diseases, neurological diseases, or inflammatory conditions, or to correct genetic defects.

Viral vector nanoparticles of the present technology include an outer shell containing a poly(beta-amino ester) polymer which encapsulates the vector. The polymer molecules are end-modified with positively charged or negatively charged oligopeptides. The polymer shell of the vector nanoparticles allows them to transduce cells without the need for pseudotyping or the inclusion of any viral fusion protein, such as VSV-G. The polymer-encapsulated vector nanoparticles have a natural tropism for peripheral blood cells, such as leucocytes, without the need for a targeting moiety, although a targeting moiety can be added for other desired target cells.

One aspect of the technology is a method of in vivo transduction of cells of a subject and expression of a transgene in the transduced cells. The method includes providing a viral vector nanoparticle that contains a viral vector lacking a viral fusion protein and encoding the transgene; and a plurality of oligopeptide modified poly(beta amino ester) (OM-PBAE) molecules forming a shell surrounding the lentiviral vector. Absence of spike proteins makes it possible for the OM-PBAE to form a complete, uninterrupted shell, thereby simplifying control over targeting, reducing immunogenicity, and improving the safety profile. The nanoparticle is administered parenterally to the subject, whereby cells of the subject are transduced by the viral vector and the transgene is expressed in the cells. The viral vector can be a lentiviral vector or another viral vector.

The method has an improved safety profile compared to a method that administers a viral vector containing a viral fusion protein and/or without a non-toxic and biodegradable polymer shell. The avoidance of a viral fusion or “spike” or pseudotyping protein in the vector, as well as encasement of the vector in a shell of the biodegradable and non-toxic OM-PBAE polymer, significantly improve the safety profile of the vector relative to a pseudotyped vector. The improved safety profile also can include one or more of the following features: reduced activation of immune cells relative to use of a pseudotyped vector, lack of change in body weight, lack of change in a blood cell count, lack of induction of a cytokine, and lack of hepatotoxicity (such as indicated by an increase in ALT/AST ratio or other changes in markers for hepatotoxicity). Lack of induction of a cytokine indicates lack of development of an immune response against the vector, such as a response that can lead to a cytokine storm. “Lack of induction of a cytokine” as used herein refers to lack of an increase in expression of a cytokine, such as one or more of IL-2, IL-4, IL-5, TNF-a, and IFN-g, as measured by plasma level of the cytokine not increasing, or increasing less than 5%, less than 10%, less than 20%, or less than 50% compared to prior to administration of the vector. Another aspect of the improved safety profile can be that the vector nanoparticles do not show tropism toward spleen, bone marrow, or liver as evaluated either by transgene expression or proviral integration. “Tropism” as used herein refers to the tendency of a viral vector to accumulate in an organ or tissue to a higher level than its average distribution in the body of a subject to whom it is administered. Yet another aspect of the improved safety profile can be that the vector nanoparticles contain OM-PBAE polymer that has been synthesized in the absence of dimethylsulfoxide (DMSO), a solvent that is undesirable for pharmaceutical formulations designed for parenteral administration.

### BRIEF DESCRIPTION OF DRAWINGS

FIGS. 1A to 1F summarize whole blood leukocyte counts after a single intravenous injection of VSV-G-deficient (“Bald”) and VSV-G+ (“pseudotyped”) lentiviral vector particles encoding for the luciferase reporter gene in Balb/c mouse model. Main leukocyte subpopulations were determined by multicolor flow cytometry 4 days before and 14 days after treatment with bald or VSV-G+ lentiviral vector particles administered at two different doses (Dose 1=2.8×10¹⁰ vector particles (vp) per mouse or Dose 2=1.4×10¹¹ vp per mouse). The fraction of blood CD3-positive lymphocytes, B lymphocytes, NK cells, monocytes/macrophages and activated lymphocytes is given for samples withdrawn 4 days before (−4) and 14 days post-treatment (14) with 2 doses of VSV-G-deficient (“Bald”) (FIG. 1A) and VSV-G+ (“pseudotyped”) lentiviral vector particles (FIG. 1B). CD4-positive T lymphocytes (FIG. 1D) and CD8-positive T lymphocytes (FIG. 1E) contents are depicted separately. Leukocyte and T lymphocytes counts 14 days post-injection are compared for the different treatments in FIGS. 1C and 1F respectively.

FIGS. 2A to 2E shows circulating cytokine levels after a single intravenous injection of VSV-G-deficient (“Bald”) and VSV-G+ (“pseudotyped”) lentiviral vector particles encoding for the luciferase reporter gene in Balb/c mouse model. Plasma levels of TNF-a (FIG. 2A), IFN-g (FIG. 2B), IL-2 (FIG. 2E), IL4 (FIG. 2D) and IL-5 (FIG. 2C) were quantified by bead-based flow cytometry method 4 days before and 14 days after treatment with bald or VSV-G+ lentiviral vector particles administered at two different doses (Dose 1=2.8×10¹⁰ vp per mouse or Dose 2=1.4×10¹¹ vp per mouse).

FIG. 3 shows results of in vivo tissue biodistribution of VSV-G-deficient (“Bald”) and VSV-G+ (“pseudotyped”) lentiviral vector particles encoding for the luciferase reporter gene in Balb/c mouse model. Whole body bioluminescence imaging was performed 3, 7 or 14 days after a single intravenous injection of bald or VSV-G+ lentiviral vector particles administered at two different doses (Dose 1=2.8×10¹⁰ vp per mouse or Dose 2=1.4×10¹¹ vp per mouse).

FIG. 4 shows results of in vivo tissue biodistribution of VSV-G-deficient (“Bald”) and VSV-G+ (“pseudotyped”) lentiviral vector particles encoding for the luciferase reporter gene in Balb/c mouse model. qPCR to detect integrated proviral sequence was performed on organs collected 14 days after a single intravenous injection of bald or VSV-G+ lentiviral vector particles administered at two different doses (Dose 1=2.8×10¹⁰ vp per mouse or Dose 2=1.4×10¹¹ vp per mouse).

FIGS. 5A to 5D summarize whole blood leukocyte counts after repeat intravenous injections of VSV-G+ (“pseudotyped”) or VSV-G-deficient (“Bald”) lentiviral vector particles encapsulated in OM-PBAE polymers encoding for the GFP and luciferase reporter genes in Balb/c mouse model. Main leukocyte subpopulations were determined by multicolor flow cytometry 8 days before and 7 days after treatment with encapsulated bald lentiviral vector particles administered via two, three, four or five intravenous doses (one per day) (total injected dose=1.3 up to 2.57×10¹¹ vector particles (vp) per mouse). VSV-G+ lentiviral vector particles were administered through five intravenous doses (total injected dose=2.57×10¹¹ vector particles (vp) per mouse). The fraction of blood CD3-positive lymphocytes, B lymphocytes, NK cells, monocytes/macrophages, neutrophils and activated lymphocytes is given for samples withdrawn 8 days before (−8) and 7 days post-treatment (7) with 2, 3, 4 or 5 doses of VSV-G-deficient particles encapsulated in OM-PBAEs (21V, 31V, 41V and 51V) and pseudotyped lentiviral vector particles (VSV-G+ 5 IV) (FIG. 5A). CD4-positive T lymphocyte and CD8-positive T lymphocyte contents are depicted separately (FIG. 5C). Leukocyte and T lymphocytes counts 7 days post-injection are compared for the different treatments in FIGS. 5B and 5D respectively.

FIGS. 6A and 6B show circulating cytokine levels after repeat intravenous injections of pseudotyped or VSV-G-deficient lentiviral vector particles encapsulated in OM-PBAE polymers encoding for the GFP and luciferase reporter genes in Balb/c mouse model. Plasma levels of TNF-a, IFN-g, IL-2, IL4 and IL-5 were quantified by bead-based flow cytometry method 8 days before and 3 (FIG. 6A) and 7 days (FIG. 6B) after treatment with 5 intravenous injections with pseudotyped (“VSV-G+”) or 2, 3, 4 or 5 intravenous injections VSV-G-deficient (“Bald”) lentiviral vector particles encapsulated in OM-PBAE polymers.

FIG. 7 shows results of in vivo tissue biodistribution after repeat intravenous injections of pseudotyped or VSV-G-deficient lentiviral vector particles encapsulated in OM-PBAE polymers encoding for the GFP and luciferase reporter genes in Balb/c mouse model. qPCR to detect integrated proviral sequence was performed on organs collected 3 days after treatment with 5 intravenous injections with pseudotyped (“VSV-G+”) or 2, 3, 4 or 5 intravenous injections VSV-G-deficient (“Bald”) lentiviral vector particles encapsulated in OM-PBAE polymers.

FIGS. 8A and 8B summarize the GFP expression profile in leukocytes after repeat intravenous injections of pseudotyped or VSV-G-deficient lentiviral vector particles encapsulated in OM-PBAE polymers encoding for the GFP and luciferase reporter genes in Balb/c mouse model. GFP-expression was measured by flow cytometry in blood, bone marrow and spleen cells collected 3 (FIG. 8A) or 7 days (FIG. 8B) after treatment with 5 intravenous injections with pseudotyped (“VSV-G+”) or 2, 3, 4 or 5 intravenous injections VSV-G-deficient (“Bald”) lentiviral vector particles encapsulated in OM-PBAE polymers.

FIG. 9 shows results of transduction (GFP expression) in different populations of mouse blood cells 3 days after treatment with each of the indicated lentiviral vectors and doses.

FIGS. 10A and 10B summarize whole blood leukocyte counts after repeated intravenous infusions or injections of VSV-G-deficient (“Bald”) lentiviral vector particles encapsulated in OM-PBAE polymers encoding for the GFP reporter genes in Balb/c mouse model. Main leukocyte subpopulations were determined by multicolor flow cytometry 9 days before, 3 and 7 days after treatment with encapsulated bald lentiviral vector particles administered via three, four or five intravenous doses (one per day) administrated either via injection or perfusion (total injected dose=1.3×10¹¹ up to 2.57×10¹¹ vector particles (vp) per mouse for the injections and =2.4×10¹¹ up to 4×10¹¹ vp per mouse for the infusions). VSV-G+ lentiviral vector particles were administered through five intravenous infusions (total injected dose=4×10¹¹ vector particles (vp) per mouse). The fraction of blood CD3-positive lymphocytes, B lymphocytes, NK cells, monocytes/macrophages, neutrophils and eosinophils is given for samples withdrawn 9 days before (−9) and 3 and 7 days post-treatment (+3 and +7) with 3, 4 or 5 injections (IV dose 1, 2 and 3) or with 3, 4 or 5 infusions (Infusion dose 1, 2 and 3) of VSV-G-deficient particles encapsulated in OM-PBAEs, with 5 perfusions of bald lentivectors (Bald) and pseudotyped lentiviral vector particles (VSV-G+) (FIG. 10A). Leucocyte counts were evaluated in mice after sacrifice (Day 7) in spleen and bone marrow and compared with whole blood leucocytes counts in FIG. 10B. A vehicle control was included in the study and administered via five infusions of 450 μL formulation buffer

FIGS. 11A and 11B summarize resident and activated myeloid whole blood counts after repeated intravenous infusions or injections of VSV-G-deficient (“Bald”) lentiviral vector particles encapsulated in OM-PBAE polymers encoding for the GFP reporter genes in Balb/c mouse model. Myeloid subpopulations were determined by multiplex flow cytometry 9 days before, 3 and 7 days after treatment with encapsulated bald lentiviral vector particles administered via three, four or five intravenous doses (one per day) administrated either via injection or perfusion (total injected dose=1.3×10¹¹ up to 2.57×10¹¹ vector particles (vp) per mouse for the injections and =2.4×10¹¹ up to 4×10¹¹ vp per mouse for the infusions). VSV-G+ lentiviral vector particles were administered through five intravenous infusions (total injected dose=4×10¹¹ vector particles (vp) per mouse). The fraction of blood resident and inflammatory myeloids cells is given for samples withdrawn 9 days before (−9) and 3 and 7 days post-treatment (+3 and +7) with 3, 4 or 5 injections (IV dose 1, 2 and 3) or with 3, 4 or 5 infusions (Infusion dose 1, 2 and 3) of VSV-G-deficient particles encapsulated in OM-PBAEs, with 5 perfusions of bald lentivectors (Bald) and pseudotyped lentiviral vector particles (VSV-G+) (FIG. 11A). Myeloid counts were evaluated in mice after sacrifice (Day 7) in spleen and bone marrow and compared with whole blood leucocytes counts in FIG. 11B. A vehicle control was included in the study and administered via five infusions of 450 μL formulation buffer.

FIGS. 12A and 12B summarize CD4+, CD8+ and TCRgamma/delta+ counts among the CD3+ cells after repeated intravenous infusions or injections of VSV-G-deficient (“Bald”) lentiviral vector particles encapsulated in OM-PBAE polymers encoding for the GFP reporter genes in Balb/c mouse model. Myeloid subpopulations were determined by multiplex flow cytometry 9 days before, 3 and 7 days after treatment with encapsulated bald lentiviral vector particles administered via three, four or five intravenous doses (one per day) administrated either via injection or perfusion (total injected dose=1.3×10¹¹ up to 2.57×10¹¹ vector particles (vp) per mouse for the injections and =2.4×10¹¹ up to 4×10¹¹ vp per mouse for the infusions). VSV-G+ lentiviral vector particles were administered through five intravenous infusions (total injected dose=4×10¹¹ vector particles (vp) per mouse). The fraction of CD3+CD4+, CD8+ and TCRgamma/delta+ cells is given for samples withdrawn 9 days before (−9) and 3 and 7 days post-treatment (+3 and +7) with 3, 4 or 5 injections (IV dose 1, 2 and 3) or with 3, 4 or 5 infusions (Infusion dose 1, 2 and 3) of VSV-G-deficient particles encapsulated in OM-PBAEs, with 5 perfusions of bald lentivectors (Bald) and pseudotyped lentiviral vector particles (VSV-G+) (FIG. 12A). CD4+, CD8+ and TCRgamma/delta+ counts among CD3+ cells were evaluated in mice after sacrifice (Day 7) in spleen and bone marrow and compared with whole blood leucocytes counts in FIG. 12B. A vehicle control was included in the study and administered via five infusions of 450 μL formulation buffer.

FIGS. 13A and 13B summarize T-Regs, naïve, central memory and effector memory counts among the CD4+ cells after repeated intravenous infusions or injections of VSV-G-deficient (“Bald”) lentiviral vector particles encapsulated in OM-PBAE polymers encoding for the GFP reporter genes in Balb/c mouse model. CD4+ subpopulations were determined by multicolor flow cytometry 9 days before, 3 and 7 days after treatment with encapsulated bald lentiviral vector particles administered via three, four or five intravenous doses (one per day) administrated either via injection or perfusion (total injected dose=1.3×10¹¹ up to 2.57×10¹¹ vector particles (vp) per mouse for the injections and =2.4×10¹¹ up to 4×10¹¹ vp per mouse for the infusions). VSV-G+ lentiviral vector particles were administered through five intravenous infusions (total injected dose=4×10¹¹ vector particles (vp) per mouse). The fraction of blood resident and inflammatory myeloid cells is given for samples withdrawn 9 days before (−9) and 3 and 7 days post-treatment (+3 and +7) with 3, 4 or 5 injections (IV dose 1, 2 and 3) or with 3, 4 or 5 infusions (Infusion dose 1, 2 and 3) of VSV-G-deficient particles encapsulated in OM-PBAEs, with 5 perfusions of bald lentivectors (Bald) and pseudotyped lentiviral vector particles (VSV-G+) (FIG. 13A). CD4+ subpopulations counts were evaluated in mice after sacrifice (Day 7) in spleen and bone marrow and compared with whole blood leucocytes counts in FIG. 13B. A vehicle control was included in the study and administered via five infusions of 450 μL formulation buffer.

FIGS. 14A and 14B summarize naïve, central memory and effector memory counts among the CD8+ cells after repeated intravenous infusions or injections of VSV-G-deficient (“Bald”) lentiviral vector particles encapsulated in OM-PBAE polymers encoding for the GFP reporter genes in Balb/c mouse model. CD8+ subpopulations were determined by multicolor flow cytometry 9 days before, 3 and 7 days after treatment with encapsulated bald lentiviral vector particles administered via three, four or five intravenous doses (one per day) administrated either via injection or perfusion (total injected dose=1.3×10¹¹ up to 2.57×10¹¹ vector particles (vp) per mouse for the injections and =2.4×10¹¹ up to 4×10¹¹ vp per mouse for the infusions). VSV-G+ lentiviral vector particles were administered through five intravenous infusions (total injected dose=4×10¹¹ vector particles (vp) per mouse). The fraction of blood resident and inflammatory myeloid cells is given for samples withdrawn 9 days before (−9) and 3 and 7 days post-treatment (+3 and +7) with 3, 4 or 5 injections (IV dose 1, 2 and 3) or with 3, 4 or 5 infusions (Infusion dose 1, 2 and 3) of VSV-G-deficient particles encapsulated in OM-PBAEs, with 5 perfusions of bald lentivectors (Bald) and pseudotyped lentiviral vector particles (VSV-G+) (FIG. 14A). CD8+ subpopulations counts were evaluated in mice after sacrifice (Day 7) in spleen and bone marrow and compared with whole blood leucocytes counts in FIG. 14B. A vehicle control was included in the study and administered via five infusions of 450 μL formulation buffer.

FIGS. 15A and 15B summarize CD25− CD69−, CD25+ CD69−, CD25− CD69+ and CD25+ CD69+ cell counts among the CD4+ cells after repeated intravenous infusions or injections of VSV-G-deficient (“Bald”) lentiviral vector particles encapsulated in OM-PBAE polymers encoding for the GFP reporter genes in Balb/c mouse model. CD4+ subpopulations were determined by multicolor flow cytometry 9 days before, 3 and 7 days after treatment with encapsulated bald lentiviral vector particles administered via three, four or five intravenous doses (one per day) administrated either via injection or perfusion (total injected dose=1.3×10¹¹ up to 2.57×10¹¹ vector particles (vp) per mouse for the injections and =2.4×10¹¹ up to 4×10¹¹ vp per mouse for the infusions). VSV-G+ lentiviral vector particles were administered through five intravenous infusions (total injected dose=4×10¹¹ vector particles (vp) per mouse). The fraction of blood resident and inflammatory myeloid cells is given for samples withdrawn 9 days before (−9) and 3 and 7 days post-treatment (+3 and +7) with 3, 4 or 5 injections (IV dose 1, 2 and 3) or with 3, 4 or 5 infusions (Infusion dose 1, 2 and 3) of VSV-G-deficient particles encapsulated in OM-PBAEs, with 5 perfusions of bald lentivectors (Bald) and pseudotyped lentiviral vector particles (VSV-G+) (FIG. 15A). Activated CD4+ subpopulations counts were evaluated in mice after sacrifice (Day 7) in spleen and bone marrow and compared with whole blood leucocytes counts in FIG. 15B. A vehicle control was included in the study and administered via five infusions of 450 μL formulation buffer.

FIGS. 16A and 16B summarize CD25− CD69−, CD25+CD69−, CD25− CD69+ and CD25+ CD69+ cell counts among the CD8+ cells after repeated intravenous infusions or injections of VSV-G-deficient (“Bald”) lentiviral vector particles encapsulated in OM-PBAE polymers encoding for the GFP reporter genes in Balb/c mouse model. CD4+ subpopulations were determined by multicolor flow cytometry 9 days before, 3 and 7 days after treatment with encapsulated bald lentiviral vector particles administered via three, four or five intravenous doses (one per day) administrated either via injection or perfusion (total injected dose=1.3×10¹¹ up to 2.57×10¹¹ vector particles (vp) per mouse for the injections and =2.4×10¹¹ up to 4×10¹¹ vp per mouse for the infusions). VSV-G+ lentiviral vector particles were administered through five intravenous infusions (total injected dose=4×10¹¹ vector particles (vp) per mouse). The fraction of blood resident and inflammatory myeloid cells is given for samples withdrawn 9 days before (−9) and 3 and 7 days post-treatment (+3 and +7) with 3, 4 or 5 injections (IV dose 1, 2 and 3) or with 3, 4 or 5 infusions (Infusion dose 1, 2 and 3) of VSV-G-deficient particles encapsulated in OM-PBAEs, with 5 perfusions of bald lentivectors (Bald) and pseudotyped lentiviral vector particles (VSV-G+) (FIG. 16A). Activated CD8+ subpopulations counts were evaluated in mice after sacrifice (Day 7) in spleen and bone marrow and compared with whole blood leucocytes counts in FIG. 16B. A vehicle control was included in the study and administered via five infusions of 450 μL formulation buffer.

FIG. 17 shows circulating cytokine levels after repeat intravenous infusions or injections of VSV-G-deficient (“Bald”) lentiviral vector particles encapsulated in OM-PBAE polymers encoding for the GFP reporter genes in Balb/c mouse model. Plasma levels of TNF-a, IFN-g, IL-2, IL4 and IL-5 were quantified by a bead-based flow cytometry method on plasma collected 9 days before and 3 and 7 days after treatment with encapsulated bald lentiviral vector particles administered via three, four or five intravenous doses (one per day) administrated either via injection or perfusion (total injected dose=1.3×10¹¹ up to 2.57×10¹¹ vector particles (vp) per mouse for the injections and =2.4×10¹¹ up to 4×10¹¹ vp per mouse for the infusions). VSV-G+ lentiviral vector particles were administered through five intravenous infusions (total injected dose=4×10¹¹ vector particles (vp) per mouse). A vehicle control was included in the study and administered via five infusions of 450 μL formulation buffer.

FIG. 18 shows plasma Aspartate Aminotransferase (AST) and Alanine Aminotransferase (ALT) enzymatic activities as biomarkers of liver failure after repeat intravenous infusions or injections of VSV-G-deficient (“Bald”) lentiviral vector particles encapsulated in OM-PBAE polymers encoding for the GFP reporter genes in Balb/c mouse model. Enzymatic activity was measured with commercial colorimetric kit on plasma collected 9 days before and 7 days after treatment with encapsulated bald lentiviral vector particles administered via three, four or five intravenous doses (one per day) administrated either via injection or perfusion (total injected dose=1.3×10¹¹ up to 2.57×10¹¹ vector particles (vp) per mouse for the injections and =2.4×10¹¹ up to 4×10¹¹ vp per mouse for the infusions). VSV-G+ lentiviral vector particles were administered through five intravenous infusions (total injected dose=4×10¹¹ vector particles (vp) per mouse). A vehicle control was included in the study and administered via five infusions of 450 μL formulation buffer.

FIG. 19 shows results of in vivo tissue biodistribution after repeat intravenous infusions or injections of VSV-G-deficient (“Bald”) lentiviral vector particles encapsulated in OM-PBAE polymers encoding for the GFP reporter genes in Balb/c mouse model. qPCR to detect integrated proviral sequence was performed on organs collected 7 days after treatment with encapsulated bald lentiviral vector particles administered via three, four or five intravenous doses (one per day) administrated either via injection or perfusion (total injected dose=1.3×10¹¹ up to 2.57×10¹¹ vector particles (vp) per mouse for the injections and =2.4×10¹¹ up to 4×10¹¹ vp per mouse for the infusions). VSV-G+ lentiviral vector particles were administered through five intravenous infusions (total injected dose=4×10¹¹ vector particles (vp) per mouse). A vehicle control was included in the study and administered via five infusions of 450 μL formulation buffer.

FIG. 20 summarizes the GFP expression profile in leukocytes after repeat intravenous infusions or injections of VSV-G-deficient (“Bald”) lentiviral vector particles encapsulated in OM-PBAE polymers encoding for the GFP reporter genes in Balb/c mouse model. GFP-expression was measured by flow cytometry in blood, bone marrow and spleen cells collected 7 days after treatment with encapsulated bald lentiviral vector particles administered via three, four or five intravenous doses (one per day) administrated either via injection or perfusion (total injected dose=1.3×10¹¹ up to 2.57×10¹¹ vector particles (vp) per mouse for the injections and =2.4×10¹¹ up to 4×10¹¹ vp per mouse for the infusions). VSV-G+ lentiviral vector particles were administered through five intravenous infusions (total injected dose=4×10¹¹ vector particles (vp) per mouse). A vehicle control was included in the study and administered via five infusions of 450 μL formulation buffer.

FIG. 21 summarizes the GFP expression profile in T lymphocyte sub-populations after repeat intravenous infusions or injections of VSV-G-deficient (“Bald”) lentiviral vector particles encapsulated in OM-PBAE polymers encoding for the GFP reporter genes in Balb/c mouse model. GFP-expression was measured by flow cytometry in blood, bone marrow and spleen cells collected 7 days after treatment with encapsulated bald lentiviral vector particles administered via three, four or five intravenous doses (one per day) administrated either via injection or perfusion (total injected dose=1.3×10¹¹ up to 2.57×10¹¹ vector particles (vp) per mouse for the injections and =2.4×10¹¹ up to 4×10¹¹ vp per mouse for the infusions). VSV-G+ lentiviral vector particles were administered through five intravenous infusions (total injected dose=4×10¹¹ vector particles (vp) per mouse). A vehicle control was included in the study and administered via five infusions of 450 μL formulation buffer.

### DETAILED DESCRIPTION

The technology described herein provides synthetic packaged viral vector nanoparticle compositions and methods for using them to provide enhanced delivery of genetic material for use in gene therapy and vaccine applications. The vector compositions and methods can be employed in any situation for which transduction of cells with one or more transgenes is useful. For example, they can be used for treatment of cancer, infectious diseases, metabolic diseases, neurological diseases, or inflammatory conditions, or to correct genetic defects.

The viral vector nanoparticle compositions of the present technology include an outer shell containing a polymer or a mixture of polymers which encapsulate the vector. In certain embodiments, the polymer shell of the nanoparticle contains one or more species of oligopeptide-derivatized poly(beta-amino ester) having the general formula

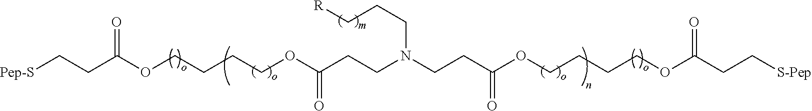

wherein Pep is a peptide, such as an oligopeptide, and R is OH, CH₃, or a cholesterol group, and wherein m ranges from 1 to 20, n ranges from 1 to 100, and o ranges from 1 to 10. In preferred embodiments, the peptide includes at least two, or at least three amino acids, selected from the group consisting of arginine (R), lysine (K), histidine (H), glutamic acid (E), aspartic acid (D), and cysteine (C). See also WO2014/136100 and WO2016/116887 for further description of oligopeptide-derivatized PBAEs which can be used in the present technology. Cysteine can be included to provide a covalent attachment point for the peptide to the polymer; while it carries a slight negative charge at pH 7, it does not significantly alter charge if used together with positively charged amino acids. Exemplary peptides are CRRR (SEQ ID NO:1), CHHH (SEQ ID NO:2), CKKK (SEQ ID NO:3), CEEE (SEQ ID NO:4), and CDDD (SEQ ID NO:5). In other embodiments, any naturally occurring amino acid can be included in the Pep moieties. The sequence of the peptide is selected so as to promote cellular uptake and targeting of the polymer-coated vector. In embodiments both utilizing and lacking viral envelope proteins, the oligopeptide sequences and net charge are selected so as to promote cellular uptake and/or endosomal uptake and/or endosomal escape of viral vectors encapsulated with the polymers in the intended target cells. Peptides at each of the two ends of the polymer typically are the same on a given polymer molecule but may be different. Polymer molecules in a mixture used to coat a vector may be the same or different; if different, they may differ in the polymer backbone or in the terminal peptides. In preferred embodiments, the peptide at both ends of the polymer has the amino acid sequence CRRR (SEQ ID NO:1), or CHHH (SEQ ID NO:2), or CKKK (SEQ ID NO:3), or CEEE (SEQ ID NO:4), or CDDD (SEQ ID NO:5). The polymer or mixture of polymers can have a net positive or negative charge, or can be uncharged. Oligopeptides containing 2 or more residues of only a single type of amino acid, such as R, K, H, D, or E, can be used. The polymer can comprise a sugar or sugar alcohol grafted onto the lateral chain attached to the polymer backbone. One or more of the polymer molecules encapsulating each vector nanoparticle can optionally comprise a targeting moiety linked to the polymer at any desired position on the polymer molecule, such as at R. The polymer molecules can be cross-linked or non-cross-linked. The polymer molecules can be attached to the viral vector surface either non-covalently, or covalently such as by covalent attachment to a lipid molecule (e.g., cholesterol, a phospholipid, or a fatty acid) that partitions into the lipid bilayer of the vector, or to a protein embedded in the lipid bilayer. In some embodiments, the polymer merely coats the vector but is attached non-specifically, i.e., it is not attached to the vector by specific covalent or non-covalent interactions, but only by nonspecific interactions such as net charge, hydrophobicity, or van der Waals interactions. In certain embodiments, the ratio of the polymer or mixture of polymers to the retroviral vector is from about 1:100 to about 5:1 by weight, or the number of polymer molecules per vector particle is from about 10⁶ to about 10¹².

The viral vector nanoparticles comprise a viral vector. The viral vector can be chosen based on desired characteristics (e.g., immunogenicity, capacity to accommodate different sized constructs, integrating and replicative properties, expression level, duration of expression, tissue tropism or targeting characteristics, ability to infect dividing and/or quiescent cells, etc.). The viral vector can be a retroviral vector such as a lentiviral vector. In preferred embodiments, the viral vector is a retroviral vector. However, the invention can also be practiced with other types of virus and/or viral vectors, including those derived from DNA or RNA viruses.

The nanoparticles can include one or more targeting moieties that are exposed at the surface of the encapsulated vector nanoparticles, such as by covalent or non-covalent association of the targeting moieties with the polymer coating. One molecular species of targeting moiety, or more than one different molecular species of targeting moiety, can be present on each viral vector nanoparticle. Targeting moieties can be, for example, antibodies, antibody fragments (including Fab), scFvs, antibody-like protein scaffolds, oligopeptides, aptamers, L-RNA aptamers, or ligands for cell surface receptors. In an embodiment, the targeting moiety is an anti-CD3 antibody or aptamer for targeting the nanoparticles towards T-cells. In an embodiment, the transgene encodes a chimeric antigen receptor. The nanoparticle is capable of acting as a gene delivery vehicle by transducing cells in vivo in a subject to whom the vehicle is administered, or transducing cells in vitro. In certain embodiments, the nanoparticle is capable of transducing a specific type of cell or class of cells, usually through the action of the targeting moiety and/or through the action of the polymer or polymer mixture. In some embodiments, the polymer of polymer-coated vector particles can promote cell transduction, not only by attachment to target cell surfaces, but through endosomal uptake, such as by endocytosis, micropinocytosis, phagocytosis, or other mechanisms, and endosomal escape (via membrane fusion after a reduction in pH in the endosomal vesicle). In preferred embodiments, the amino acid sequence of oligopeptides associated with the polymer can specifically promote cell transduction via the endosomal route.

While in some embodiments of the present technology the viral vectors are pseudotyped, in other embodiments the vectors lack certain viral or pseudotyping envelope proteins. In pseudotyped vectors, envelope proteins such as the HIV gp120-gp41 complex or the vesicular stomatitis virus (VSV-G) glycoprotein form spike-like structures on the outer surface of the viral envelope, which facilitate attachment of the vector particles to host cells and entry of viruses into host cells. Pseudotyping proteins such as VSV-G also can be used to protect or bind the vector during purification (e.g., protection during ultracentrifugation, binding to an affinity column or other affinity matrix, or gel purification). However, in the context of polymer packaging of a viral vector, such structures can interfere with tight association between the vector particle envelope and the polymers. Viral envelope proteins also carry a pH-dependent charge, which can limit or interfere with the association of polymers, particularly charged polymers. Further, packaged viral vectors harboring pseudotyping proteins can undergo coating destabilization and destruction in vitro and in vivo, thus releasing viral vector able to transduce cells nonspecifically, potentially leading to a reduction in safety profile. Vectors lacking envelope proteins can enhance the packaging of viral vectors using polymers. Coated vectors lacking envelope protein show an improved safety profile compared to vectors having envelope protein, because after destabilization or destruction of the coating, they are not able to transduce cells in vitro. The nanoparticles of the present technology wherein the viral vector lacks viral envelope protein have a built-in safety mechanism for use in gene therapy or immunotherapy, because the nanoparticles are only capable of transducing a mammalian cell above a threshold number of polymer molecules per vector. Below that threshold number of polymer molecules per vector, the amount of polymer is insufficient to completely coat the vector, which can cause the nanoparticles to become structurally unstable or subject to dissociation of polymer molecules from the nanoparticle. Once such vectors lacking envelope protein lose an effective polymer coating, they become incapable of transducing mammalian cells, including human cells. Such nanoparticles have an improved safety profile for in vivo use compared to a vector or polymer-encapsulated vector that lacks the threshold feature.

In some embodiments, some membrane proteins, such as proteins that are not viral envelope or fusion proteins and do not otherwise form spike-like structures on the membrane outer surface, such as proteins from the vector producing cells not used for pseudotyping, may be present in the lipid membrane of the viral vector particle. In certain embodiments of the present technology, viral envelope proteins are excluded from the viral vector particles, such as those which have a significant mass protruding from the outer surface of the envelope lipid bilayer, such as at least 20%, at least 30%, at least 40%, or at least 50% of their mass protruding from the outer envelope surface.

The nanoparticle compositions optionally can contain additional components, such as lipid molecules, surfactants, nucleic acids, protein molecules, or small molecule drugs. The present technology also contemplates pharmaceutical formulations or compositions containing the nanoparticles together with one or more excipients, carriers, buffers, salts, or liquids, rendering the delivery vehicle suitable for administration via oral, intranasal, or parenteral administration, such as intravenous, intramuscular, subcutaneous, peritumoral or intratumoral injection, or for in vitro administration to cells in an ex vivo gene transfer protocol. Such compositions and formulations can also be lyophilized to stabilize them during storage.

The viral vector nanoparticle of the present technology can serve as a gene delivery vehicle. The nanoparticle includes a viral vector coated on its exterior surface with a layer containing a polymer or a mixture of polymers. In some embodiments, the viral vector is pseudotyped and possesses a fusion-promoting envelope protein and comprises a transgene. In other embodiments, the viral vector lacks any native or recombinant envelope protein and comprises a transgene. In certain embodiments, the retroviral vector specifically lacks the viral envelope protein that might typically be included in the envelope of similar retroviral vectors. In certain embodiments, the viral vector specifically lacks any naturally occurring or modified viral vector envelope proteins, such as wild type or modified VSV-G, HIV gp120, HIV gp41, MMTV gp52, MMTV gp36, MLV gp71, syncytin, wild type or modified Sindbis virus envelope protein, measles virus hemagglutinin (H) and fusion (F) glycoproteins, and HEMO. In other embodiments, the vector contains one or more of such envelope proteins.

Another aspect of the technology is a method of making the above-described nanoparticle/gene delivery vehicle (viral vector nanoparticle). The method includes the steps of: (a) providing a viral vector either possessing or lacking envelope protein and containing a transgene; (b) providing a polymer or a mixture of polymers; and (c) contacting the viral vector and the polymer or mixture of polymers, whereby the viral vector and the polymer/mixture of polymers combine to form the nanoparticle, which contains the viral vector coated with the polymer or mixture of polymers.

A further aspect of the technology is an in vivo method of treating a disease using the above-described viral vector nanoparticles (gene delivery vehicles). The method requires parenterally administering a composition containing the nanoparticles to a subject in need thereof, whereby cells within the subject are transduced by the viral vector and the transgene is expressed in the transduced cells. In one embodiment, the disease to be treated is cancer. For in vivo administration, embodiments in which viral envelope proteins such as VSV-G are absent are preferred, as they provide an enhanced safety profile because they lack the ability to transduce non-targeted cells in the host and due to more specific targeting provided by the use of polymer encapsulation to restore cellular uptake and/or endosomal uptake and/or endosomal escape otherwise provided by envelope protein.

### Example 1. Production of Batches of Lentiviral Vectors Used in Biodistribution Studies

Transduction-deficient lentiviral vectors lacking the fusogenic and highly immunogenic VSV-G protein, which were previously engineered (see WO 2019/145796 A2, which is hereby incorporated by reference), were prepared for use in biodistribution studies in mice. These vectors allow repeat systemic administration.

The different batches of lentiviral vectors injected in a healthy mouse model to follow the tissue biodistribution of the transgene were made using the following materials and methods.

Materials

The transfer vector plasmid was pARA-CMV-GFP or pARA-hUBC-Luciferase or pAra-hUBC-Luciferase-T2A-GFP. A kanamycin-resistant plasmid encoded for the provirus (a non-pathogenic and non-replicative recombinant proviral DNA derived from HIV-1, strain NL4-3), in which an expression cassette was cloned. The insert contained the transgene, the promoter for transgene expression and sequences added to increase the transgene expression and to allow the lentiviral vector to transduce all cell types including non-mitotic ones. The coding sequences corresponded to the gene encoding Green Fluorescent Protein (GFP) or firefly Luciferase (bioluminescent reporter protein) or the bi-cistronic cassette that drives the concomitant expression of both Luciferase and GFP transgenes separated by the self-cleaving 2A peptide sequence. The promoter was the human ubiquitin promoter (hUBC) or the CMV promoter. It was devoid of any enhancer sequence and it promoted gene expression at a high level in a ubiquitous manner. The non-coding sequences and expression signals corresponded to Long Terminal Repeat sequences (LTR) with the whole cis-active elements for the 5′LTR (U3-R-U5) and the deleted one for the 3′LTR, hence lacking the promoter region (ΔU3-R-U5). For the transcription and integration experiments, encapsidation sequences (SD and 5′Gag), the central PolyPurine Tract/Central Termination Site for the nuclear translocation of the vectors, and the BGH polyadenylation site were added.

The packaging plasmid was pARA-Pack. The kanamycin resistant plasmid encoded for the structural lentiviral proteins (GAG, POL, TAT and REV) used in trans for the encapsidation of the lentiviral provirus. The coding sequences corresponded to a polycistronic gene gag-pol-tat-rev, coding for the structural (Matrix MA, Capsid CA and Nucleocapside NC), enzymatic (Protease PR, Integrase IN and Reverse Transcriptase RT) and regulatory (TAT and REV) proteins. The non-coding sequences and expression signals corresponded to a minimal promoter from CMV for transcription initiation, a polyadenylation signal from the insulin gene for transcription termination, and an HIV-1 Rev Responsive Element (RRE) participating for the nuclear export of the packaging RNA.

The envelope plasmid, when used, was pENV1. This kanamycin-resistant plasmid encoded glycoprotein G from the Vesicular Stomatitis Virus (VSV-G) Indiana strain, used for the pseudotyping of some of the lentiviral vectors. The VSV-G genes were codon optimized for expression in human cells, and the gene was cloned into pVAX1 plasmid (Invitrogen). The coding sequences corresponded to codon-optimized VSV-G gene, and the noncoding sequences and expression signals corresponded to a minimal promoter from CMV for transcription initiation, and the BGH polyadenylation site to stabilize the RNA.

Production of VSV-G⁻ (“Bald”) Lentiviral Vector Particles

LV293 cells were seeded at 5×10⁵ cells/mL in 2×3000 mL Erlenmeyer flasks (Corning) in 1000 mL of LVmax Production Medium (Gibco Invitrogen). The two Erlenmeyers were incubated at 37° C., 65 rpm under humidified 8% CO₂. The day after seeding, the transient transfection was performed. PEIPro transfectant reagent (PolyPlus Transfection, Illkirch, France) was mixed with transfer vector plasmid (pARA-CMV-GFP or pARA-hUBC-Luciferase or pARA-hUBC-Luciferase-T2A-GFP) and packaging plasmid (pARA-Pack). After incubation at room temperature, the mix PEIPro/Plasmid was added dropwise to the cell line and incubated at 37° C., 65 rpm under humidified 8% CO₂. At day 3, the lentivector production was stimulated by sodium butyrate at 5 mM final concentration. The bulk mixture was incubated at 37° C., 65 rpm under humidified 8% CO₂ for 24 hours. After clarification by deep filtration at 5 and 0.5 μm (Pall Corporation), the clarified bulk mixture was incubated 1 hour at room temperature for DNase treatment.

Lentivector purification was performed by chromatography on a Q mustang membrane (Pall Corporation) and eluted by NaCl gradient. Tangential flow filtration was performed on a 100 kDa HYDROSORT membrane (Sartorius), which allowed to reduce the volume and to formulate in specific buffer at pH 7, ensuring at least 2 years of stability. After sterile filtration at 0.22 μm (Millipore), the bulk drug product was filled in 2 mL glass vials with aliquots less than 1 mL, then labelled, frozen and stored at <−70° C.

The bald LV number was evaluated by physical titer quantification. The assay was performed by detection and quantitation of the lentivirus associated HIV-1 p24 core protein only (Cell Biolabs Inc.). A pre-treatment of the samples allows to distinguish the free p24 from destroyed Lentivectors. Physical titer, particle distribution and size were measured by tunable resistive pulse sensor (TRPS) technology (qNano instrument, Izon Science, Oxford, UK). NP150 nanopore, 110 nm calibration beads and membrane stretch between 44 and 47 mm were used. The results were determined using the IZON Control Suite software.

Production of VSV-G⁺ (“Pseudotyped”) Lentiviral Vector Particles

The same above-described method was used except that PEI Pro transfectant reagent (PolyPlus, 115-010) was mixed with transfer vector plasmid (pARA-CMV-GFP or pARA-hUBC-Luciferase or pARA-hUBC-Luciferase-T2A-GFP), packaging plasmid (pARA-Pack) and the envelope plasmid (pENV1).

Production, Purification and Quantification of OM-PBAE Polymer—Classical Method

Poly (β-amino ester)s (PBAEs) were prepared in a two-step procedure as described by Dosta et al. with slight modifications. First step is the synthesis of PBAE-diacrylate polymers, and the second step comprises the synthesis of peptide modified PBAEs (OM-PBAE) in DMSO.

Synthesis, Purification and Characterization of PBAE-Diacrylate Polymers

Poly (β-amino ester)-diacrylate polymer was synthesized via addition type polymerization using primary amine and diacrylate functional monomers. 5-amino-1-pentanol (Sigma-Aldrich, 95.7% purity, 3.9 g, 36.2 mmol), 1-Hexylamine (Sigma-Aldrich, 99.9 purity, 3.8 g, 38 mmol) and 1,4-butanediol diacrylate (Sigma-Aldrich, 89.1% purity, 18 g, 81 mmol) were mixed in a round bottom flask at molar ratio of 2.2:1, acrylate to primary amine groups. The mixture was stirred at 90° C. for 20 h. Then the crude product, a light-yellow viscous oil, was obtained by cooling the reaction mixture to room temperature and stored at −20° C. until further use.

Synthesized PBAE-diacrylate polymers were characterized using 1H-NMR spectroscopy to confirm the structures and GPC to determine the molecular weight characteristics. NMR spectra were collected in Bruker 400 MHz Avance III NMR spectrometer, with 5 mm PABBO BB Probe, Bruker and DMSO-d6 was used as deuterated solvent. Molecular weight determination was conducted on a Waters HPLC system equipped with a GPC SHODEX KF-603 column (6.0× about 150 mm), and THF as mobile phase and with an RI detector. The molecular weights were determined using a conventional calibration curve obtained by polystyrene standards. Weight average molecular weight (M_{w}) and number average molecular weight (Mₙ) of crude PBAE-diacrylate polymer were determined as 4900 g/mol and 2900 g/mol, respectively.

Synthesis, Purification and Characterization of OM-PBAEs in DMSO

OM-PBAE polymers were obtained by peptide end-modification of PBAE-diacrylate polymers via thiol-acrylate Michael addition reaction in DMSO at a thiol/diacrylate ratio of 2.8:1. Synthesis of tri-arginine modified PBAE polymer (PBAE-CR3) is given as an example: crude PBAE-diacrylate polymer (199 mg, 0.08 mmol) was dissolved in DMSO (1.1 mL) and a hydrochloride salt of NH₂—Cys-Arg-Arg-Arg-COOH peptide (CR3—95% purity—purchased from Ontores Biotechnologies, Zhejiang, China) (168 mg, 0.23 mmol) was dissolved in DMSO (1 mL). Then the solutions of polymer and peptide were mixed and stirred at 25° C. in a temperature-controlled water bath for 20 h. Peptide modified PBAE was precipitated in 20 ml diethylether/acetone (7/3, v/v), then the product was washed two times with 7.5 mL diethylether/acetone (7/3, v/v), followed by vacuum drying and resulting product was resuspended in DMSO at a concentration of 100 mg/ml and stored at −20° C. for further use.

In a further example, tri-histidine end-modified PBAE polymer, PBAE-CH3, the solution of PBAE-diacrylate (199 mg, 0.08 mmol) dissolved in DMSO (1.1 mL) and mixed with a solution of hydrochloride salt of NH₂—Cys-His-His-His-COOH (CH3) (154 mg, 0.23 mmol) in DMSO (1.0 mL).

1H-NMR analysis confirmed the expected structures. Further, the percentage of acrylate conversion was determined from the ratio of CH₃ protons (0.8 ppm) on the polymer backbone which was calibrated to the same value as in the spectrum of the starting material to residual acrylate peaks (5.75-6.5 ppm). Therefore, dividing the integration value of acrylate peaks to six (which is the number of protons on the acrylate groups) yielded the residual acrylate amount. The Michael addition reaction efficiencies for peptide modified PBAEs from crude PBAE-diacrylate were determined as; PBAE-CR3: 84% and PBAE-CH3: 93%. However, in both cases overall yields of the reactions were greater than 100% indicating the presence of a large excess of residual DMSO. Furthermore, the residual peptide content in each peptide modified PBAE was quantified by UV detection (wavelength 220 nm) after separation by UPLC ACQUITY system (Waters) equipped with a BEH C18 column (130 A, 1.7 μm, 2.1×50 mm, temperature 35° C.) using an acetonitrile/water with 0.1% TFA as gradient.

Production, Purification and Quantification of OM-PBAE Polymer—DMSO-Free Method

The gram-scale synthesis of functional OM-PBAEs in DMSO-free conditions has been previously established in order to provide new agents for gene delivery that are not toxic to human cells. Polymers obtained with this proprietary method contain less impurities and they can be stably stored in conditions that are compatible with biological systems.

Synthesis, Purification and Characterization of PBAE-Diacrylate Polymers

After the synthesis of PBAE-diacrylate polymers was performed as previously described, the reaction mixture was purified by heptane precipitation. Crude product was dissolved in ethyl acetate and added dropwise into excess heptane (1/10, v/v), this procedure being repeated twice. Purified PBAE-diacrylate was obtained with an 86% yield and characterized by GPC to have M_{w} and Mₙ, 5200 g/mol and 3300 g/mol, respectively. Moreover, GPC curves for crude PBAE-diacrylate obtained by classical method and purified PBAE-diacrylate polymers demonstrated that the small peaks at the low molecular weight region on the GPC trace of the crude product disappeared after the purification and the peak molecular weight moved slightly to a higher value (4900 and 5000, for crude and purified polymer, respectively).

Synthesis, Purification and Characterization of OM-PBAEs in DMSO-Free Conditions

1 g scale synthesis of OM-PBAEs was performed in acetonitrile/citrate (25 mM, pH 5.0) (3/2, v/v) using purified PBAE-diacrylate precursor polymer and 2× concentrated peptide solution. Inert nitrogen (N₂) atmosphere was applied during the reaction to prevent di-sulfide formation in the reaction medium. Synthesis of tri-arginine modified PBAE polymer (PBAE-CR3) is given as an example. Purified PBAE-diacrylate polymer (1999 mg, 0.624 mmol) was dissolved in acetonitrile (20 mL) and a hydrochloride salt of NH₂—Cys-Arg-Arg-Arg-COOH peptide (CR3-97% purity—purchased from Ontores) (1684 mg, 2.3 mmol) was dissolved in citrate buffer (25 mM, pH 5.0) (20 mL), after complete dissolution of peptide 10 ml acetonitrile was added. Then the solution of polymer in acetonitrile was added to the peptide solution in citrate (25 mM, pH 5.0)/acetonitrile (2/1, v/v) and stirred at 25° C. in a temperature-controlled water bath for 20 h under N₂ atmosphere. Then, all the solvents were evaporated at 40° C. under reduced pressure. Resulting pellet was extracted with 100 mL of ethanol, twice. Ethanol extracts were dried. The dry rest was re-dissolved in 50 mL of ethanol and precipitated in 200 ml diethylether/acetone (7/3, v/v), then product was washed two times with 75 mL diethylether/acetone (7/3, v/v). Residual organic solvents were removed under vacuum, further final product was obtained by lyophilization with a 37% (wt %) yield and stored at −20° C. for further use.

For tri-histidine end modified PBAE polymer (PBAE-CH3), purified PBAE-diacrylate polymer (1999 mg, 0.624 mmol) was dissolved in acetonitrile (20 ml) and a hydrochloride salt of NH2-Cys-His-His-His-COOH peptide (CH3—98% purity—purchased from Ontores) (1.538 mg, 2.3 mmol) was dissolved in 20 mL 25 mM citrate buffer at pH 5.0. After complete dissolution of CH3 peptide 10 mL acetonitrile was added. Then the solution of polymer in acetonitrile was added to the peptide solution in acetonitrile/citrate (25 mM, pH 5.0) (2/1, v/v) and stirred at 25° C. in a temperature-controlled water bath for 20 h under inert N₂ atmosphere. Then, all the solvents were evaporated at 40° C. under reduced pressure. Resulting pellet was extracted with 100 mL of ethanol, twice. Ethanol extracts were dried. The dry rest was re-dissolved in 50 mL of ethanol and precipitated in 200 mL diethylether/acetone (7/3, v/v), then product was washed two times with 75 mL diethylether/acetone (7/3, v/v). Residual organic solvents were removed under vacuum, further final product was obtained with a 45.3% (wt %) yield and stored at −20° C. for further use.

Coating of VSV-G⁻ (“Bald”) Lentiviral Vectors with Oligopeptide-Modified PBAE

For intravenous injections, coating of lentiviral vectors (4.5 to 5.1×10¹⁰ lentiviral viral particles) was performed with a ratio of 10⁹ polymer molecules per lentiviral vector particle as follows. Bald lentiviral vectors were diluted in Dulbecco's phosphate-buffered saline (DPBS) (Gibco Invitrogen) containing 50 mM Sucrose (Sigma-Aldrich) to prepare a final volume of 75 μL per replicate. R and H OM-PBAE polymers (with or without DMSO) previously mixed 60/40 (v/v) were diluted in 25 mM calcium citrate buffer pH 5.4 (75 μL per replicate) and vortexed 2 s for homogenization. The diluted polymers were added to the diluted vectors in a 1:1 ratio (v/v), the mixes were gently vortexed for 10 s and incubated 10 minutes at room temperature. Finally, an equal volume of 25 mM calcium citrate buffer pH 5.4 (150 μL) was added to the coated particles before injection.

For perfusions, the same protocol was used but volumes were adjusted to larger volumes as follows. Bald lentiviral vectors (8×10¹⁰ lentiviral viral particles) were diluted in (DPBS) containing 50 mM Sucrose to prepare a final volume of 225 μL per replicate. R and H OM-PBAE polymers previously mixed 60/40 (v/v) were diluted in 25 mM calcium citrate buffer pH 5.4 (164 μL per replicate) and vortexed 2 s for homogenization. Finally, volume was adjusted with 25 mM calcium citrate buffer pH 5.4 (61 μL) added to the coated particles before injection.

Coating of VSV-G⁻ (“Bald”) Lentiviral Vectors with Oligopeptide-Modified PBAE Using a Microfluidics-Based Process

In order to tightly control the homogeneity, monodispersity of VSV-G− (“Bald”) lentiviral vector particles encapsulated in OM-PBAEs and allow batch-to-batch consistency during the manufacturing, a microfluidics-based encapsulation process was implemented.

Bald lentiviral vectors were diluted in Dulbecco's phosphate-buffered saline (DPBS) (Gibco Invitrogen) containing 50 mM sucrose (Sigma-Aldrich) to the appropriate concentration. R and H OM-PBAE polymers (with or without DMSO) previously mixed 60/40 (v/v) were diluted in 25 mM calcium citrate buffer pH 5.4 (75 μL per replicate) and vortexed 2 s for homogenization. Each solution was injected with a TYGON tube (internal diameter=0.02 inch and outer di=0.06 inch) connected to a Y-shaped inox inlet of the microfluidics chamber. Encapsulation of VSV-G-deficient LVs (“bald”) with OM-PBAE polymers was performed within a sterile environment in 30 min at room temperature by mixing both components in a polydimethylsiloxane (PDMS) serpentine chamber (geometry length=18 cm×width=400 μm×height=100 μm) under controlled pressure conditions (Fluigent) (pressure difference between chamber inlet and outlet of 25 mbar).

This method allowed the fast and robust preparation of monodispersed and homogenous nanoparticles as shown by reference biophysical methods (Nanoparticle Tracking Analysis, Dynamic Light Scattering, Videodrop). Before systemic administration to animals, the functionality of the nanoparticles was verified in vitro in transduction cell assays.

### Example 2. In Vivo Tissue Biodistribution of VSV-G⁻ (“Bald”) and VSV-G⁺ (“Pseudotyped”) Lentiviral Vector Particles

Animal Experiments

Animal experiments were performed in accordance with the regulations of the French animal protection law and the respective European Union guidelines after having obtained formal approval of the institutional and the national animal care committee.

4-6 weeks-old female Balb/c mice (Janvier Labs, Le Genest-Saint-Isle, France) were allowed to adapt to the animal facility for 2 weeks before being anesthetized with 2% isoflurane and intravenously injected (tail vein) with a single dose of VSV-G⁻ (“Bald”) or VSV-G+(“pseudotyped”) Lentiviral Vector Particles encoding for luciferase under the control of the hUBC promoter formulated in DPBS-50 mM sucrose. Two doses of 1.4×10¹¹ lentiviral viral particles in 250 μL (corresponding to the Maximum Administered Dose or “MAD”) or 2.8×10¹⁰ lentiviral viral particles in 250 μL (MAD/5) were tested on 3 mice per condition. Behavior of animals, body weight, water and food consumption were recorded 3 times a week over a period of 14 days.

Blood Cell Count

Blood cell count was determined on fresh and heparinized whole blood samples collected from Balb/c mice 4 days before treatment (submandibular sampling) or 14 days after the intravenous injection of the lentiviral vector particles by cardiac puncture on animals anesthetized with 2% isoflurane. Blood cells were incubated with mouse Fc Block reagent (BD Biosciences). Phenotypes of circulating cells were analyzed by flow cytometry (AttuneNXT; Invitrogen, Inc.) with specific antibody panels purchased from Miltenyi Biotec: general panel (CD45-APC, CD3e-PerCP-Vio700, CD45R(B220)-PE-Vio615, CD11b-PE, CD49b-PE-Vio770), activated T-cells (CD45-APC, CD4-PercP-Vio700, CD69-PE, CD8a-PE-Vio615 and CD25-PE-Vio770) or myeloid cells (CD45-APC, CD11b-PE-Vio615, Ly-6G-PerCP-Vio700, F4/80-PE and CD11c-PE-Vio770). After 10 min incubation at 4° C., red blood cells were lysed at room temperature with RBC lysis buffer (Invitrogen Inc.). Cells were centrifuged at 500 g for 2 min and fixed with CellFix solution (BD Biosciences). Fluorescence-positive cells were counted by flow cytometry (AttuneNXT; Invitrogen, Inc.) on BL3 (PerCP-Vio700 dye), YL1 (PE dye), YL2 (PE-Vio-615 dye) and YL4 (PE-Vio770 dye) channels. Cell phenotypes were defined among CD45+, viable and single cells as follows: T lymphocytes (CD3e^{high}-BB220^{neg}), B lymphocytes (BB220^{high}) and NK cells (CD49^{high}-CD11b^{high}) on the general panel; CD4+T lymphocytes (CD4^{high}) and CD8⁺ T lymphocytes (CD4^{high}) on the activated T-cells panel; neutrophils (Ly6^{high}), monocytes (Ly6^{low}-CD11c^{low}-CD11b^{high}) and macrophages (CD11c^{low}-CD11b^{high}-F4/80^{high}) on the myeloid cells panel.

Cytokine Profiling

Plasma was harvested from fresh peripheral blood of mice collected before treatment or 14 days after intravenous injection of the lentiviral vector particles by centrifugation for 10 min at 1500× g at room temperature. Supernatant was transferred into fresh tubes and centrifuged again for 15 min at 2,000×g. Plasma was stored at −80° C. until analysis with Mouse Th1/Th2 Cytometric Bead Array (CBA) (Becton Dickinson Biosciences) following the manufacturer's instructions. Samples were analyzed by flow cytometry (AttuneNXT; Invitrogen, Inc.) on the YL-1 channel and plasma levels of secreted Interleukin 2 (IL-2), Interleukin 4 (IL-4), Interleukin 5 (IL-5), Tumor Necrosis Factor alpha (TNF-a) and Interferon gamma (IFN-g) were quantified with the AttuneNXT software (Invitrogen, Inc.).

During the two week observation period, no obvious sign of toxicity was observed. None of the treated mice experienced body weight loss, distress or behavioral change after a single bolus injection of pseudotyped lentiviral vectors or their VSV-G-deficient engineered variants.

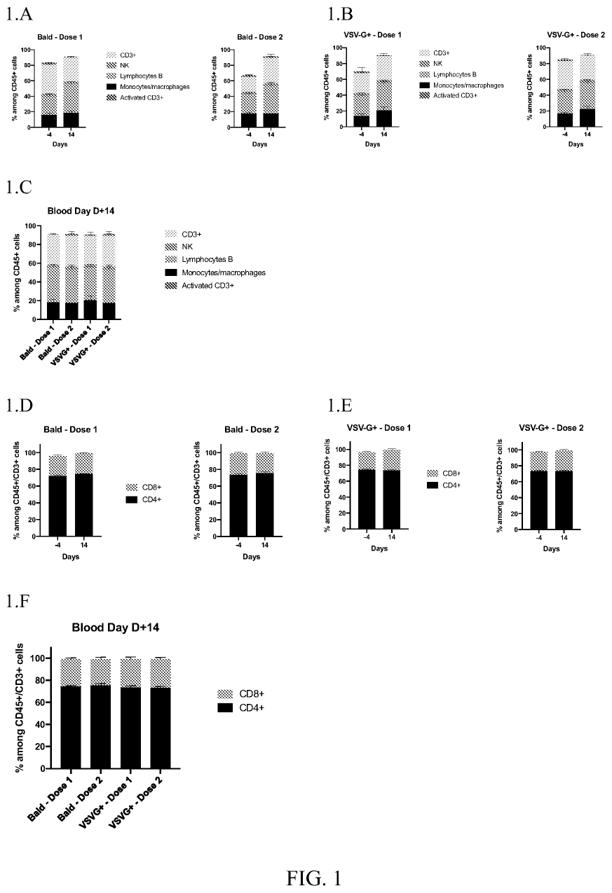

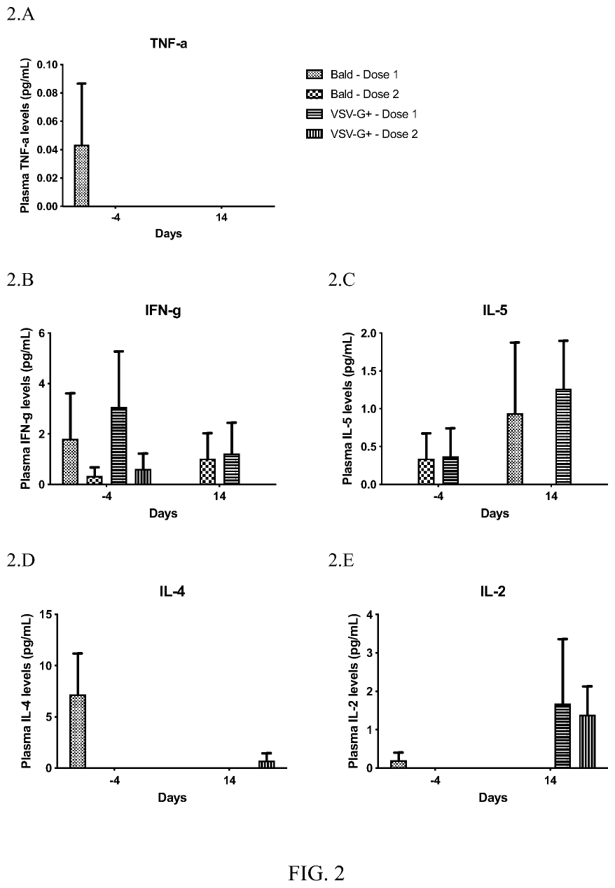

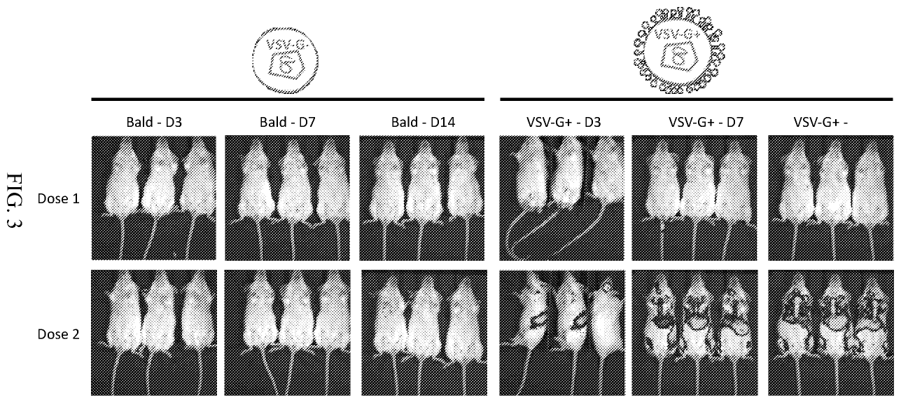

As shown in FIGS. 1A-1F, no change in blood leucocyte composition was observed 14 days after treatment compared to blood composition determined on untreated animals. Bald or pseudotyped lentviral vector did not induce any leukopenia, monocytosis or lymphocytosis, lymphodepletion, T cell activation suggesting no acute effect on the immune system. This lack of activation of the immune system was further confirmed by plasmatic levels of cytokine levels depicted in FIGS. 2A-2F. The highest doses of both types of lentiviral vectors did not induce any significant up-regulation of pro-inflammatory mediators or cytokines involved in the activation of T lymphocytes.

These results indicate that engineered VSV-G-deficient lentiviral vectors showed a similar safety profile compared to pseudotyped particles and were well tolerated after a single bolus intravenous injection.

In Vivo Bioluminescense Imaging

To follow up tissue distribution of Lentiviral Vector Particles, IVIS imaging was performed on days 3, 7 and 14 post-intravenous injection. For this purpose, Balb/c mice anesthetized with 2% isoflurane (Forane, Baxter Healthcare) were intraperitoneally injected with D-luciferin (Perkin Elmer) in PBS (15 mg/mL) at 150 mg/kg body weight.

Imaging data were obtained 10 min after D-luciferin injection with a Xenogen IVIS Spectrum Imaging System (Xenogen). Acquisition times ranged from 10 s to 3 min. Living Image software version 4.3.1 (Xenogen) was used to acquire and quantitate the data.

Results summarized in FIG. 3 show that after a single intravenous injection of the highest dose of pseudotyped lentiviral particles, a bioluminescence signal reflecting the localization of the expression of luciferase reporter gene accumulated in a time-dependent manner from 3 days post-treatment in the liver, the spleen and bone marrow (spine and lower limbs). In contrast, no transgene expression was observed in the whole body of mice injected with equivalent doses of VSV-G-deficient lentiviral vectors. These unexpected observations suggest that the removal of the VSV-G protein deeply modify the biodistribution profile of the engineered lentiviral vectors that become unable to transduce cells in vivo.

Evaluation of Tissue Distribution by Quantitative PCR

14 days post-injection of the Lentiviral Vector Particles, heparinized whole blood was sampled by cardiac puncture from Balb/c mice anesthetized with 2% isoflurane. Mice were then sacrificed by cervical dislocation and the following organs were collected: spleen, bone marrow (flushed from tibial bone with DPBS), liver, lymph nodes lung, muscle, kidney, small intestine, gonads and brain. Genomic DNA was isolated from fresh blood and frozen tissues using Nucleospin 8 Blood and Nucleospin Tissue kits (Macherey-Nagel) respectively and according to the manufacturer's instructions.

Biodistribution of lentiviral vectors was analyzed by tracking the integration of the pARA backbone in genomic DNA isolated from the indicated tissues. Vector copy number (VCN) per ng DNA analysis was performed by quantitative PCR (qPCR) using PerfeCTa® MultiPlex ToughMix® reagent (Quantabio, Beverly, Mass., USA) and CFX96 real-time PCR Instrument II (Biorad). Data were analyzed with CFX Manager 3.1 Software. Integrated lentiviral vector detection was performed using the Long Terminal Repeat sequences (LTR)-specific probe (5′-6FAM-AACCATTAGGAGTAGCACCCACCAAGG-BHQ1-3′)(SEQ ID NO:6) and primers (fwd: 5′-TGGAGGAGGAGATATGAGGG-3′ (SEQ ID NO:7) and rev: 5′-CTGCTGCACTATACCAGACA-3′) (SEQ ID NO:8) (Sigma-Aldrich). As an internal reference, a mouse GAPDH-specific probe (5′-Cy5-CGCCTGGTCACCAGGGCTGC-BHQ2-3′) (SEQ ID NO:9) and primers (fwd: 5′-AACGGATTTGGCCGTATTGG-3′ (SEQ ID NO:10) and rev: 5′-CATTCTCGGCCTTGACTGTG-3′) (SEQ ID NO:11) were used. A plasmid standard containing sequence of LTR and mouse GAPDH was used for quantification.

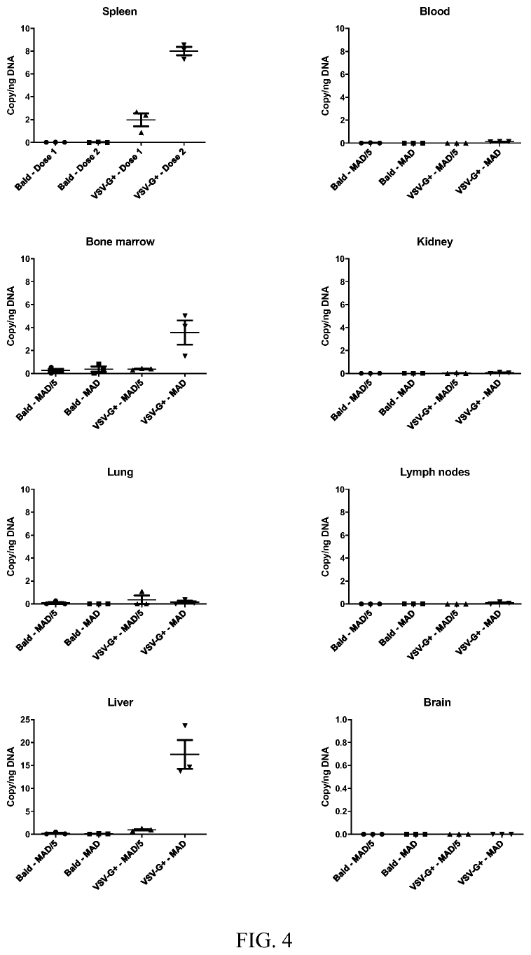

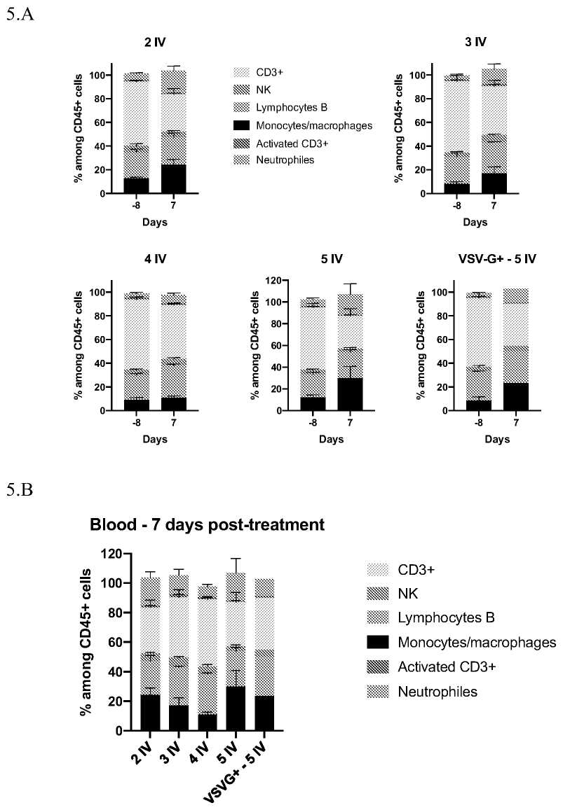

qPCR analysis of biodistribution of pseudotyped lentiviral vectors perfectly correlated with in vivo bioluminescence imaging results as integrated proviral sequences were detected in spleen, liver, bone marrow collected on mice that received the highest dose of viral particles as depicted in FIG. 4. VSV-G-deficient lentiviral vectors were not detected in any of the analyzed organs, which confirms their fundamentally different biodistribution profile and inability to integrate within the host genome.

### Example 3. In Vivo Evaluation of Safety and Biodistribution of VSV-G⁻ (“Bald”) Encapsulated in OM-PBAEs and VSV-G⁺ (“Pseudotyped”) Lentiviral Vector Particles after Repeat Intravenous Infection

To rule out that the unexpected biodistribution of VSV-G-deficient lentiviral vectors was not due to a loss of function of their gene transfer machinery, a follow-up study was performed with these engineered vectors but encapsulated in OM-PBAE polymers previously described to restore their transduction properties. In addition, this protocol aimed at assessing the safety of repeat intravenous dosing of these nanoparticles.

Animal Experiments

Animal experiments were performed in accordance with the regulations of the French animal protection law and the respective European Union guidelines after having obtained formal approval of the institutional and the national animal care committee.

4-6 week-old female Balb/c mice (Janvier Labs, Le Genest-Saint-Isle, France) were allowed to adapt to the animal facility for 2 weeks before being anesthetized with 2% isoflurane and intravenously injected (tail vein) with doses of VSV-G⁻ (“Bald”) encapsulated in OM-PBAEs as described in Example 1 or VSV-G⁺ (“pseudotyped”) lentiviral vector particles encoding for luciferase-T2-GFP under the control of the hUBC promoter formulated in DPBS-50 mM sucrose. Groups of 3 animals received 2, 3, 4 or 5 repeat intravenous injections (one per day) of 5.1×10¹¹ lentiviral viral particles in 220 μL (corresponding to total doses of 1.3 up to 2.57×10¹¹ lentiviral particles per mouse, i.e., 2/5, 3/5, 4/5 of the Maximum Administered Dose or “MAD”). Behavior of animals, body weight, water and food consumption were recorded 3 times a week over a period of 7 days.

Blood Cell Count

Blood cell count was determined as already described in Example 2 on fresh and heparinized whole blood samples collected from Balb/c mice 8 days before treatment (submandibular sampling) or 6 h, 3 days and 7 days after the last intravenous injection of the lentiviral vector particles by cardiac puncture on animals anesthetized with 2% isoflurane.

Cytokine Profiling

Circulating levels were quantified as already described in Example 2 with plasma harvested from fresh peripheral blood of mice collected before treatment or 6 h, 3 or 7 days after the last intravenous injection of the lentiviral vector particles.

During the one-week observation period, no obvious sign of toxicity was observed. None of the treated mice experienced body weight loss, distress or behavioral change after repeat injection of pseudotyped lentiviral vectors or the VSV-G-deficient engineered variants encapsulated in OM-PBAEs. In this protocol, repeat anesthesia required for intravenous dosing turned out to be the most challenging procedure and required a careful monitoring of the mice during the wakening phase.

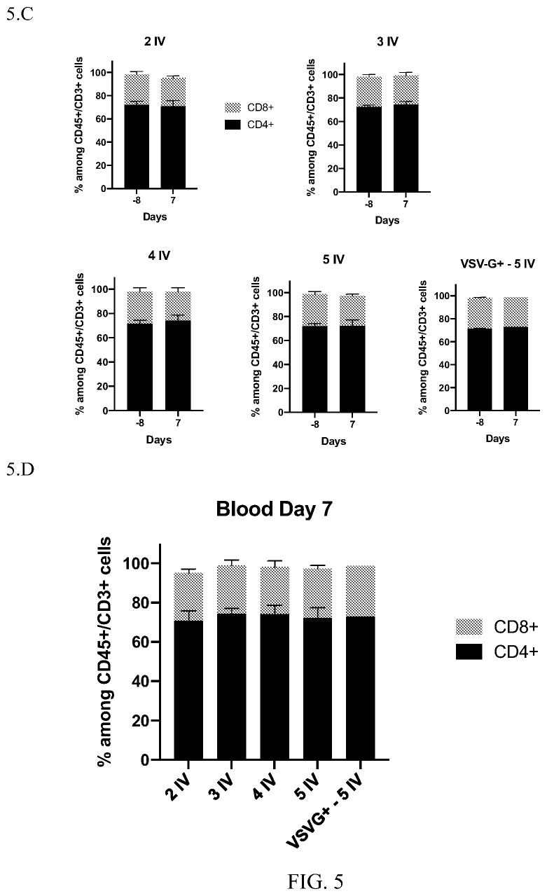

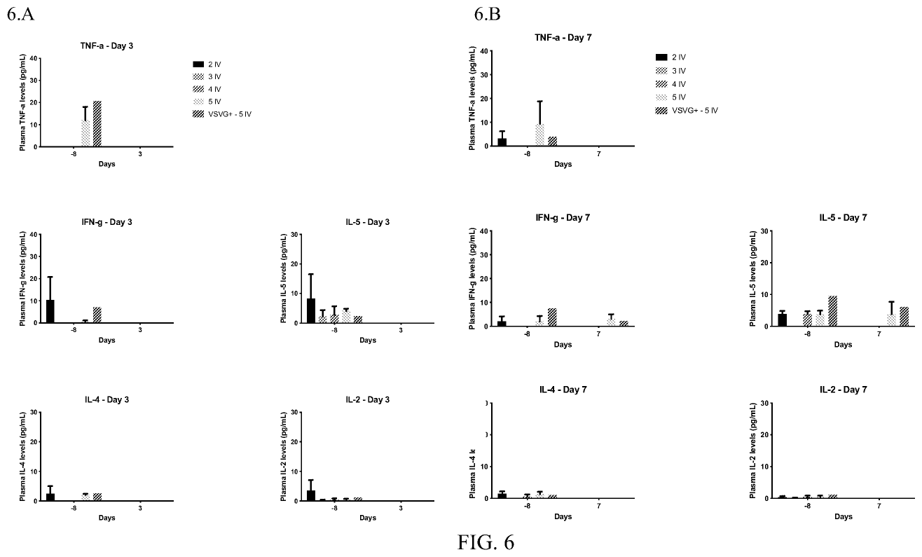

As shown in FIGS. 5A-5D, an increase in monocytes/macrophages and neutrophils counts and decrease in T lymphocytes were observed across all groups 3 or 7 days after treatment compared to blood composition determined on untreated animals. These effects were not dose-dependent and were already in place 6 h post-injection, suggesting that they were not product-related. Indeed, repeat Isoflurane anesthesia have been shown to induce modifications of blood cell counts, including reduction of the T lymphocyte population (Stolling et al., 2016). Nevertheless, VSV-G-deficient engineered variants encapsulated in OM-PBAEs or pseudotyped lentiviral vector did not induce any leukopenia or T cell activation suggesting no acute effect on the immune system. This lack of activation of the immune system was further confirmed by plasmatic levels of cytokine levels depicted in FIGS. 6A-6B. The highest doses of both types of lentiviral vectors did not induce any up-regulation of pro-inflammatory mediators or cytokines involved in the activation of T lymphocytes.

These results indicate that engineered VSV-G-deficient lentiviral vectors encapsulated in OM-PBAE polymers showed a similar safety profile compared to pseudotyped particles and were well tolerated after repeat intravenous injections.

In Vivo Bioluminescence Imaging

Tissue distribution of pseudotyped lentiviral vectors and VSV-G-deficient lentiviral vector encapsulated in OM-PBAES by IVIS imaging was carried out generally as described in Example 2 except that image acquisition was performed 3 or 7 days after the last intravenous injection of the products.

As previously described, pseudotyped lentiviral particles produced bioluminescent signals released upon expression of the luciferase reporter gene that localized in a time-dependent manner from 3 days post-treatment in the liver, the spleen and bone marrow (spine and lower limbs). In contrast, no transgene expression localized to any particular organ was observed in mice injected with equivalent doses of VSV-G-deficient lentiviral vectors encapsulated in OM-PBAE polymers. The different applied doses of nanoparticles induced diffuse signals distributed across the whole body.

Evaluation of Tissue Distribution by Quantitative PCR

Analysis of genome integration of proviral sequences delivered by pseudotyped or VSV-G-deficient lentiviral vector particles encapsulated in OM-PBAES was carried out generally as described in Example 2 except that qPCR was performed on genomic DNA extracted from blood and tissues collected 6 h, 3 or 7 days after the last intravenous injection of the products.

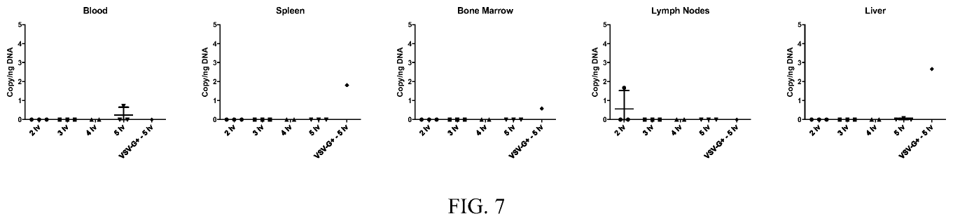

Again, qPCR analysis of biodistribution of pseudotyped lentiviral vectors perfectly correlated with in vivo bioluminescence imaging results as integrated proviral sequences were detected in spleen, liver, bone marrow collected on mice three days after the last intravenous injections as depicted in FIG. 7. VSV-G-deficient lentiviral vectors encapsulated in OM-PBAE polymers were not detected in any of the analyzed organs but in blood cells at the highest administered dose. Integration in lymph nodes was detected in one mouse treated with the lowest dose. No integration was detected in the liver which is in line with the ability of OM-PBAE-based nanoparticles to bypass liver uptake and increase (Brugada-Vila et al. 2020). If OM-PBAEs have been shown to improve blood persistence of nanoparticles, the in vivo transduction of blood quiescent cells without prior activation of their proliferation is unexpected.

Evaluation of Cellular GFP Expression by Flow Cytometry

Fresh and heparinized whole blood samples were collected from Balb/c mice 7 days before treatment (submandibular sampling) or 6 h, 3 days and 7 days after the last intravenous injection of the lentiviral vector particles by cardiac puncture on animals anesthetized with 2% isoflurane. Moreover, spleen and bone marrow were collected at sacrifice. Single cell suspensions were obtained by meshing the tissues through a 100 μm cell strainer followed by a red blood cells lysis step according to protocol for dissociation of lymphoid tissues provided with RBC lysis buffer (Invitrogen Inc.).

The percentage of blood circulating cells expressing GFP was determined by flow cytometry with antibody panels previously described for leukocyte counts and recording the GFP fluorescence with the BL1 channel. Additionally, the phenotype of transduced cells expressing GFP transgene in the blood, bone marrow and spleen was determined by co-staining with different antibodies specific for the following cell types following manufacturer's instructions (BD Biosciences): CD3 (CD3e-BB700), CD4 (CD4-PE), CD8 (CD8a-PE-Cy5.5) for T lymphocytes and CD19 (CD19-PE-CF594) for B lymphocytes. Cells were fixed with CelIFix solution (BD Biosciences) and the fluorescence-positive cells were counted by flow cytometry (AttuneNXT; Invitrogen, Inc.) on BL1 (GFP), YL1 (PE dye), YL2 (PE-CF594) or YL3 (PE-Cy5.5) channels.

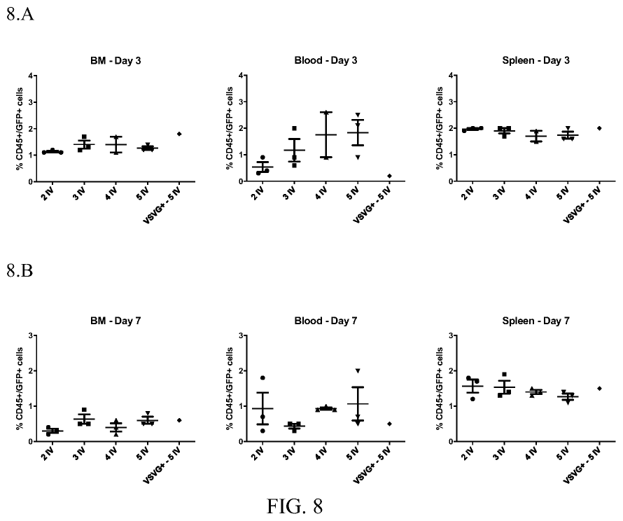

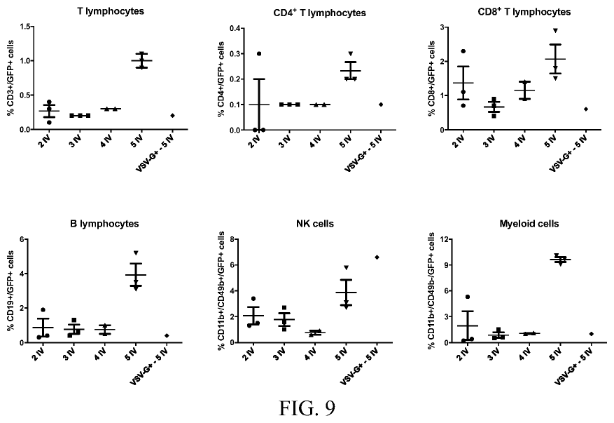

As shown in FIG. 8, GFP-positive leukocytes were found in the bone marrow and spleen of mice treated with pseudotyped lentiviral particles that were unable to transduce blood cells, thereby confirming previous bioluminescence and qPCR results. In contrast VSV-G-deficient lentiviral vectors encapsulated in OM-PBAE polymers induced GFP-expression in a dose-dependent manned in blood cells from 3 days post-treatment. Further analysis of the transduced leukocyte sub-populations revealed that all main cell types could be efficiently transduced with the nanoparticles as depicted in FIG. 9.

All together these results suggest that VSV-G-deficient lentiviral vectors encapsulated in OM-PBAE polymers are fundamentally different from their pseudotyped counterparts and have an unexpected tropism for blood cells. These are able to transduce in vivo all leukocytes subpopulation and deliver a transgene without the need of any targeting agent or prior activation of proliferation that is normally required with lentiviral-mediated gene transfer.

### Example 4. In Vivo Evaluation of Safety and Biodistribution of VSV-G⁻ (“Bald”) Lentiviral Vector Particles Encapsulated in DMSO-Free OM-PBAEs after Repeat Intravenous Injection or Infusion

An animal experiment was carried out with DMSO-free OM-PBAE polymers to test a formulation of VSV-G-deficient lentiviral vectors that comply with pharmaceutical requirements for parenteral administration.

In clinical practice, marketed gene (ZOLGENSMA®) and CAR-T cell (TECARTUS®, KYMRIAH®, YESCARTA®) therapies are currently administered to the patients via infusions. Therefore, this study compared the safety and potential of repeat infusions to deliver higher doses of lentiviral vectors over intravenous administrations.

Animal Experiments

Balb/c mice underwent the same procedures and treatments as already described in Example 3. Doses of VSV-G⁻ (“Bald”) lentiviral vector particles encapsulated in OM-PBAEs as described in Example 1 encoding for GFP under the control of the CMV promoter were intravenously injected (tail vein) within one minute under general anesthesia with 2% isoflurane. Groups of 3 animals received 3, 4 or 5 repeat intravenous injections (one per day) of 4.5×10¹¹ lentiviral viral particles in 250 μL (corresponding to total doses of 1.3 up to 2.2×10¹¹ lentiviral particles per mouse, i.e 0.3, 0.4 or 0.5 of the MAD). Doses of VSV-G⁻ (“Bald”) encapsulated in OM-PBAEs or VSV-G⁺ (“pseudotyped”) lentiviral vector particles encoding for GFP under the control of the CMV promoter formulated in DPBS-50 mM sucrose were infused (tail vein) for 20 min at a controlled flow of 22.5 μL/min. Groups of 3 animals received 3, 4 or 5 repeat infusions (one per day) of 8.1×10¹¹ lentiviral viral particles in 450 μL (corresponding to total doses of 2.4 up to 4×10¹¹ lentiviral particles per mouse, i.e 0.6, 0.8 or MAD). Control groups were included with animals treated with 5 repeat infusions (one per day) of vehicle (DPBS/calcium citrate formulation buffer), VSV-G⁻ (“Bald”) (8×10¹⁰ lentiviral viral particles in 450 μL corresponding to a total dose of 4×10¹¹ lentiviral particles per mouse, i.e MAD) or VSV-G⁺ (“pseudotyped”) lentiviral vector (3.1×10¹⁰ lentiviral viral particles in 450 μL corresponding to a total dose 1.5×10¹¹ lentiviral particles per mouse, i.e 0.4 MAD). Behavior of animals, body weight, water and food consumption were recorded 3 times a week over a period of 7 days.

Blood, Spleen and Bone Marrow Cell Count

Blood cell count was determined as already described in Example 2 on fresh and heparinized whole blood samples collected from Balb/c mice 9 days before treatment or 3 days (submandibular sampling) and 7 days after the last intravenous injection or infusion of the lentiviral vector particles by cardiac puncture on animals anesthetized with 2% isoflurane. Spleen and bone marrow were collected at sacrifice. Single cell suspensions were obtained by meshing the tissues through a 100 μm cell strainer followed by a red blood cells lysis step according to protocol for dissociation of lymphoid tissues provided with RBC lysis buffer (Invitrogen Inc.).

Different antibody panels were designed in order to gain more insight on the effect of the treatments on myeloid cells, CD4+ and CD8+ T lymphocytes composition. Cell phenotype were analyzed by flow cytometry (AttuneNXT; Invitrogen, Inc.) with specific antibody panels purchased from Biolegend: general panel (CD45-AF700, CD170(Siglec-F)-PE, Ly-6G-PerCP-Cy5.5, CD335-PE-Dazzle594, CD45R(B220)-PE-Cy7, CD3e-APC, F4/80-BV421, CD11b-BV510, CD11b-BV510, CD11b-BV510, Ly6C-BV605 and CD11c-BV711) and T cell panel (CD45-AF700, CD8-PerCP-Cy5.5, CD25-PE, CD69-PE-Dazzle594, CD62L-PE-Cy7, CD3e-APC, CD127-BV421, CD4-BV510, TCRgd-BV605, CD44-BV711).

Blood, spleen and bone marrow cells were incubated 5 min with mouse Fc Block reagent (BD Biosciences). After 20 min incubation at 4° C. with antibodies, red blood cells were lysed at room temperature with RBC lysis buffer (Invitrogen Inc.). Cells were centrifuged at 500 g for 2 min and fixed with CellFix solution (BD Biosciences).

Fluorescence-positive cells were counted by flow cytometry (AttuneNXT; Invitrogen, Inc.) BL3 (PerCP-Cy5 dye), YL1 (PE dye), YL2 (PE-Dazzle dye), YL4 (PE-Cy7 dye), RL1 (APC dye), RL2 (AF700 dye), RL3 (APC-Cy7 dye), VL1 (BV421 dye), VL2 (BV510 dye), VL3 (BV605 dye) and VL4 (BV711 dye) channels.

Cell phenotypes were defined among CD45⁺, viable and single cells as follows: T lymphocytes (CD3e^{pos}-BB220^{neg}), B lymphocytes (CD3e^{neg}-BB220^{high}) and NK cells (CD3e^{neg}-BB220^{neg}-CD11c^{neg}-CD11b^{low}-CD335^{pos}), neutrophils (SCC^{high}-CD170^{pos}-Ly6^{high}), eosinophils (CD170^{high}-Ly6^{high}), monocytes/macrophages (CD11c^{high}-CD11b^{high}-F4/80^{low/high}), resident monocytes/macrophages (CD11c^{high}-CD11b^{high}-Ly6C^{neg}) and inflammatory monocytes/macrophages (CD11c^{high}-CD11b^{high}-Ly6C^{pos}) on the general panel; T lymphocytes (CD3e^{pos}), CD4⁺ T lymphocytes (CD4^{pos}), CD8⁺ T lymphocytes (CD8^{pos}), gamma/delta T lymphocytes (CD3^{pos}-TCRgd^{pos}), Treg CD4+ lymphocytes (CD4^{pos}-CD25^{high} CD127^{low}), naïve CD4+ lymphocytes (CD4^{pos}-CD44^{low}-CD62L^{high}), central memory CD4+ lymphocytes (CD4^{pos}-CD44^{high}-CD62L^{high}), effector memory CD4+ lymphocytes (CD4^{pos}-CD44^{high}-CD62L^{neg}), naïve CD8+ lymphocytes (CD8^{high}-CD44^{low}-CD62L^{high}), central memory CD8+ lymphocytes (CD8^{pos}-CD44^{high}-CD62L^{high}), effector memory CD8+ lymphocytes (CD8^{pos}-CD44^{high}-CD62L^{neg}), activated CD4 lymphocytes (CD4^{high}-CD25^{neg/high}-CD69^{neg/high}) and activated CD8 lymphocytes (CD8^{high}-CD25^{neg/high}-CD69^{neg/high}) on the T lymphocyte panel.

Cytokine Profiling

Circulating levels were quantified as already described in Example 2 and 3 with plasma harvested from fresh peripheral blood of mice collected 9 days before treatment or 3 or 7 days after the last intravenous injection or infusion of the lentiviral vector particles.

Plasma AST/ALT Activity Measurement

Plasma was harvested from fresh peripheral blood of mice collected before treatment or 7 days after the last intravenous injection or infusion of the lentiviral vector particles by centrifugation for 10 min at 1500×g at room temperature. Supernatant was transferred into fresh tubes and centrifuged again for 15 min at 2,000×g. Plasma was stored at −80° C. until measurement of Aspartate Aminotransferase (AST) and Alanine Aminotransferase (ALT) enzymatic activities on undiluted samples with AST and ALT activity colorimetric assays (Merck-Millipore) following manufacturer's instructions.

During the one-week observation period, no obvious sign of toxicity was observed. None of the treated mice experienced body weight loss, distress or behavioral change after repeat injection or infusion of bald, pseudotyped lentiviral vectors or the VSV-G-deficient engineered variants encapsulated in OM-PBAEs. As already observed in Example 3, repeat anesthesia required for dosing turned out to be the most challenging procedure and required a careful monitoring of the mice during the wakening phase.

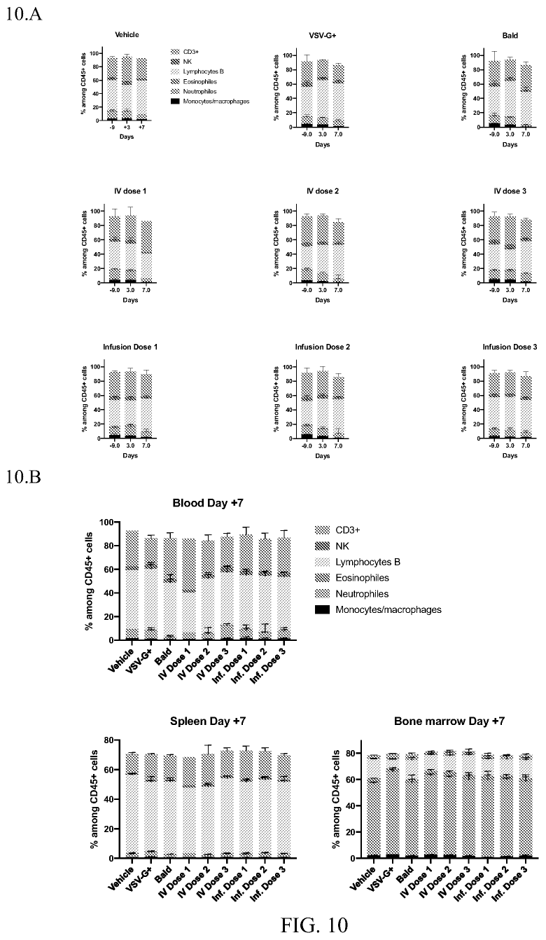

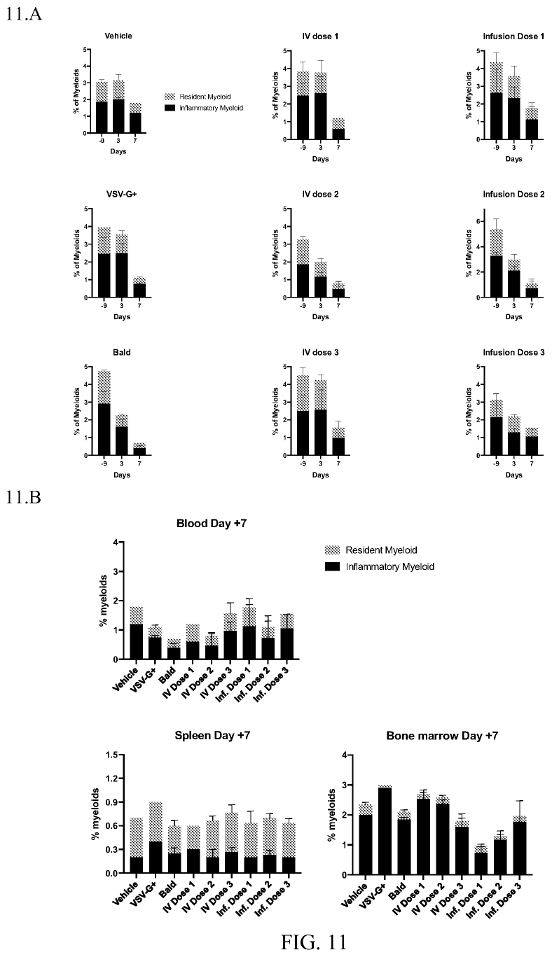

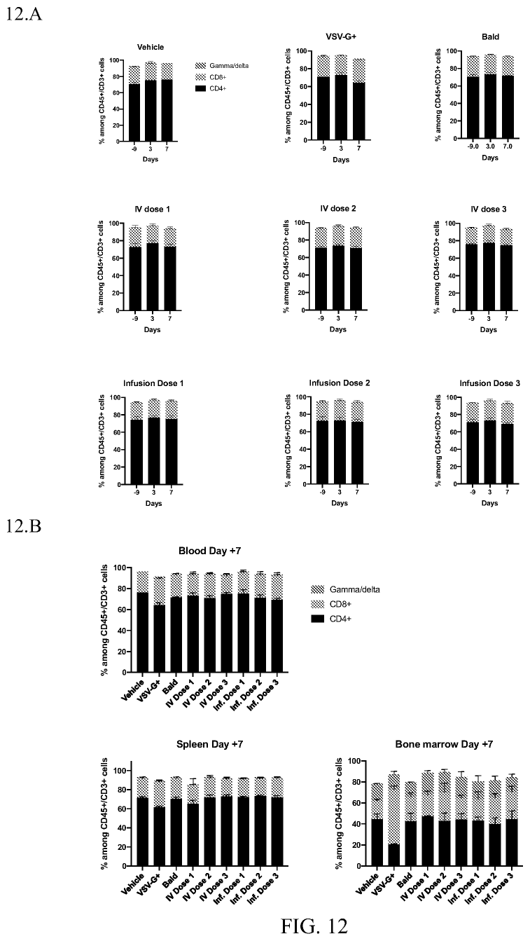

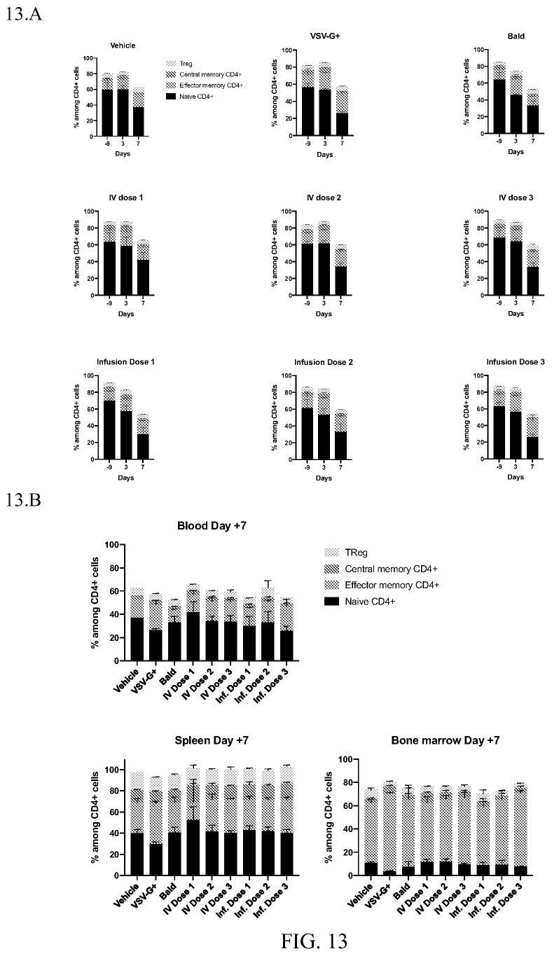

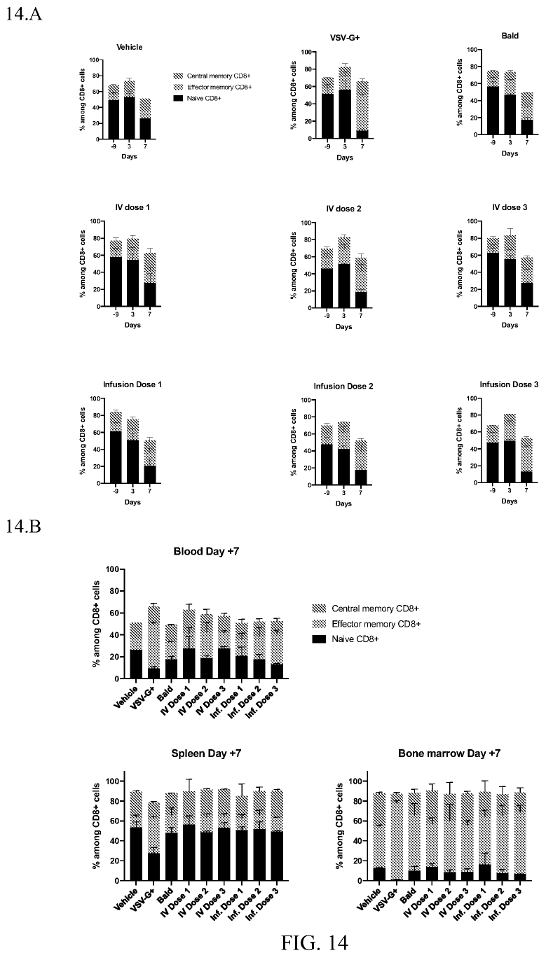

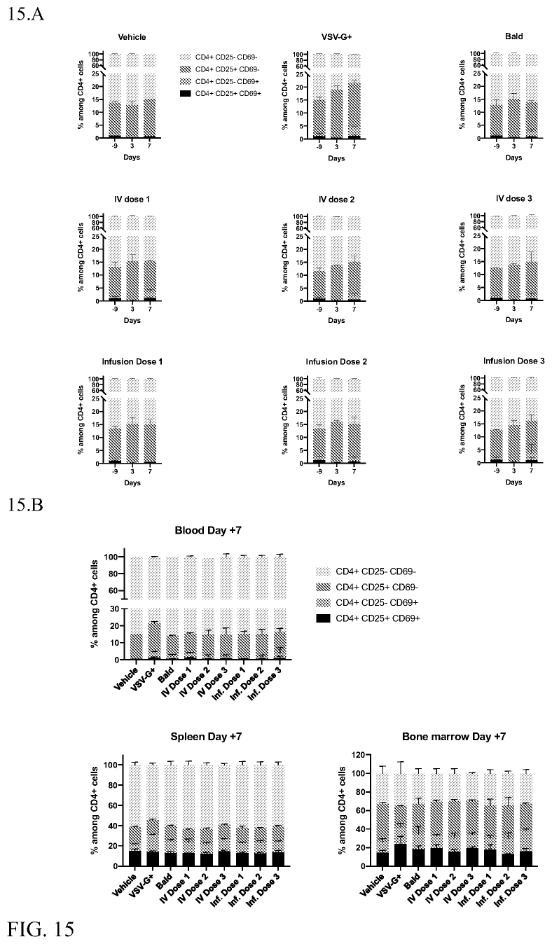

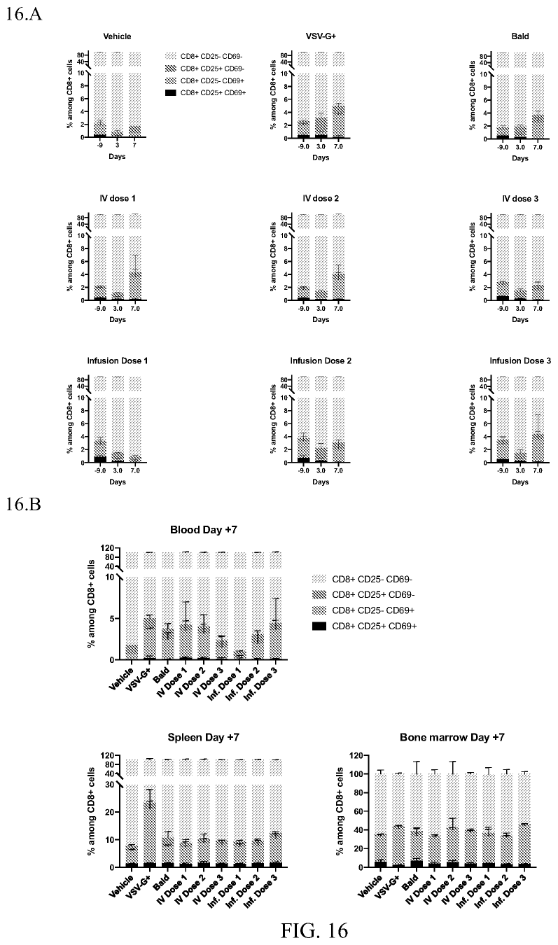

As shown in FIGS. 10A and 10B, the different treatments did not cause any significant modification of the general leukocyte composition in blood, spleen or bone marrow during the 7 days observation period. All treated animals exhibited a time-dependent reduction in blood monocytes/macrophages and macrophages whereas an increase in neutrophils was observed in the bone marrow of mice injected with pseudotyped lentiviral vectors. A focus on the myeloid population revealed that all animals experienced a pronounced drop in the number of resident and inflammatory cells, probably as a consequence of repeat of anesthesia. Interestingly as depicted in FIG. 11A, no difference in blood cell count was visible after 7 days recovery in animals injected with VSV-G-deficient engineered variants encapsulated in OM-PBAEs whereas a dose-dependent reduction in inflammatory myeloid cells was observed in the bone marrow. However, pseudotyped lentiviral vectors (FIG. 11B) caused a more pronounced effect with an increase of inflammatory myeloid cells in the spleen and bone marrow. Repeat intravenous injections or infusions of the encapsulated lentiviral vectors had no effect on CD4+, CD8+, or gamma/delta T lymphocytes that remained constant in the blood, spleen and bone marrow during the 7 days period (FIGS. 12A and 12B). Pseudotyped vectors induced an increase in the CD8-positive and a decrease in the CD4-positive lymphocytes. Within the CD4- and CD8-positive subpopulations, all treatments induced a time-dependent reduction of naïve cells (FIGS. 13A and 14A). After 7 days recovery, no difference in CD4- and CD8-positive composition was seen in blood, spleen and bone marrow from mice treated with VSV-G-deficient engineered variants encapsulated in OM-PBAEs (FIGS. 13B and 14B). Pseudotyped lentiviral vectors induced a decrease in both naïve CD4+ and CD8+ cells associated with an increase in the CD4-positive and CD8-positive effector/memory cells in blood, spleen and bone marrow. Finally, the expression of CD25 and CD69 activation markers in CD4-positive T lymphocytes was not up-regulated in groups treated with VSV-G-deficient engineered variants encapsulated in OM-PBAEs (FIGS. 15A and 15B). However, an increase in CD8⁺CD25⁻CD69⁺ cells was visible at day seven reflecting a temporary activation status, CD69 being described as an early activation marker. Pseudotyped lentiviral vectors increased CD4⁺CD25⁺CD69⁻ cells in the blood CD25⁻CD69⁺ in the spleen and bone marrow and (FIG. 15B) together with a higher occurrence of CD8⁺CD25⁻CD69⁺ cells in the spleen and bone marrow and a reduction of CD8⁺CD25⁻CD69⁺ only in the bone marrow (FIG. 16B). This detailed analysis of the phenotype of blood, spleen and bone marrow leukocytes suggests that repeat infusion of pseudotyped lentiviral vectors triggers early events of innate and adaptive immune responses against the highly immunogenic VSV-G protein within 7 days post-treatment. None of these molecular and cellular events were observed in animals that received repeat intravenous and infusions of VSV-G-deficient lentiviral particles encapsulated in DMSO-free OM-PBAEs. Despite the presence of OM-PBAE polymers containing charged tetrapeptides on their surface, these nanoparticles appear to have a favorable safety profile and are compatible with repeat treatment cycles.

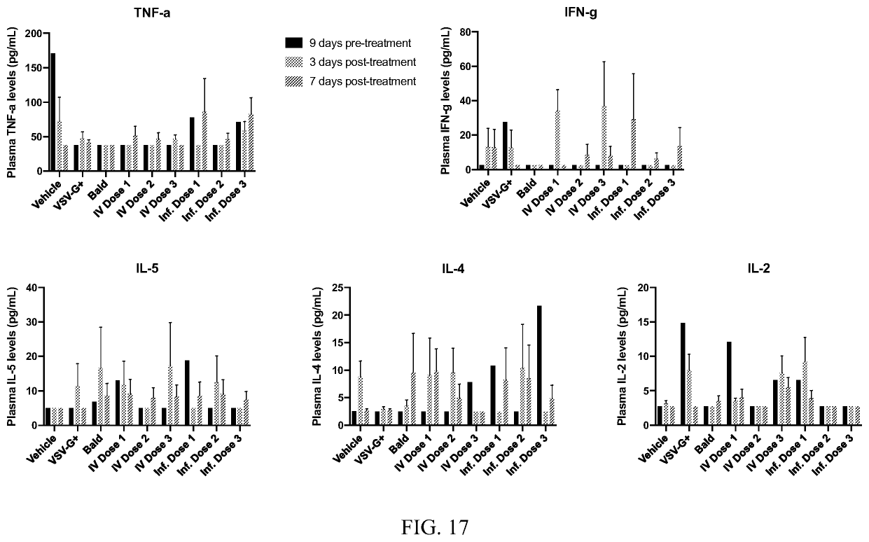

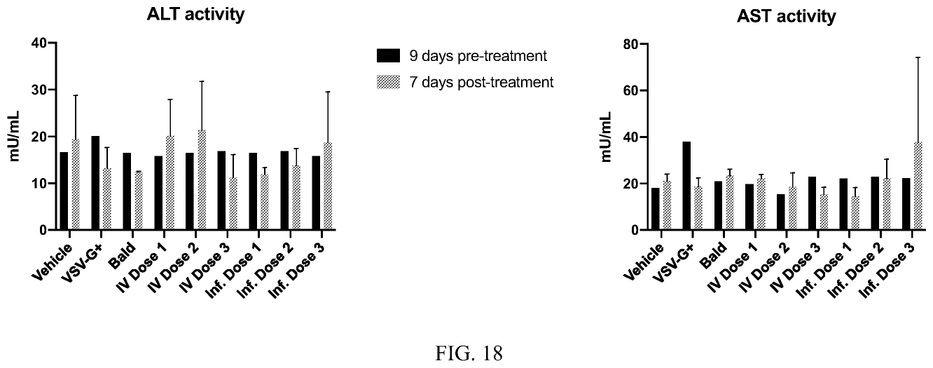

This lack of activation of the immune system was further confirmed by plasma cytokine levels as depicted in FIG. 17. During the one-week observation period, no obvious sign of cytokine upregulation was observed in all treated mice. Despite early signs of innate and adaptive immune responses against pseudotyped lentiviral vectors, levels of the tested cytokines were not higher than background levels measured in control animals injected with vehicle. The highest doses of VSV-G-deficient engineered variants encapsulated in DMSO-free OM-PBAEE did not induce any up-regulation of pro-inflammatory mediators or cytokines involved in the activation of T lymphocytes. Temporary elevated secretions of IFN-g, IL-4 and IL-5 were observed after 3 days, but plasma levels went back to background levels.

These results indicate that engineered VSV-G-deficient lentiviral vectors encapsulated in DMSO-free OM-PBAE polymers showed a favorable safety profile and were well tolerated after repeat intravenous injections or infusions.

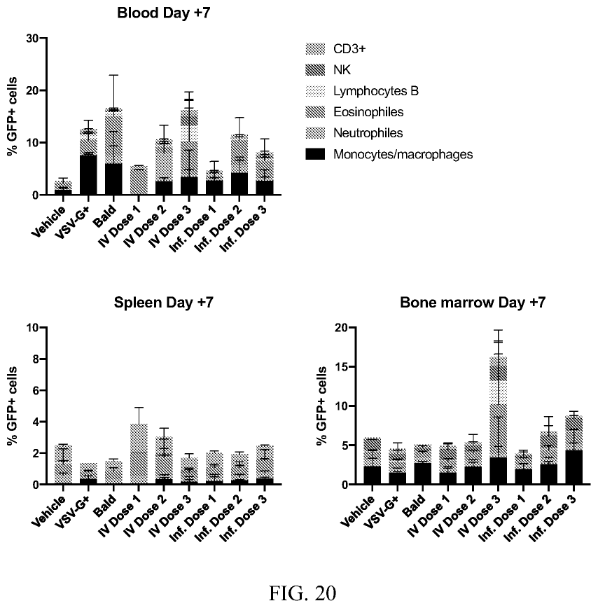

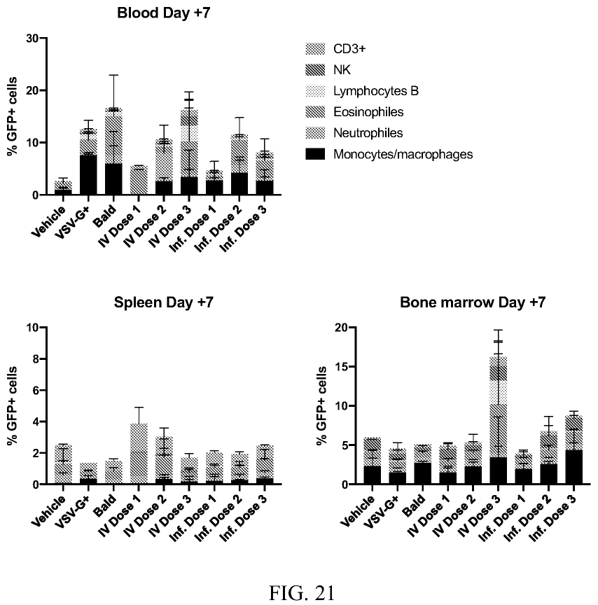

Finally, the impact of the different treatments on liver function was investigated as this organ is the primary site of drug detoxification and metabolism. Aspartate Aminotransferase (AST) and Alanine Aminotransferase (ALT) are hepatic enzymes involved metabolic reactions that are released in the blood stream upon liver injury or failure. AST and ALT enzymatic activities can be measured in the plasma with commercial reagents. FIG. 20 shows that during the one-week observation period, no obvious sign of hepatoxicity was observed in all treated mice. AST and ALT activities were not different from the values obtained before treatment and all fell within normal ranges described for normal healthy mice (ALT: 25-60 mU/mL; AST: 50-100 mU/mL). These results confirm that repeat administrations of pseudotyped, bald lentiviral vectors and the VSV-G-deficient engineered variants encapsulated in OM-PBAE do not cause acute liver damages.

Evaluation of Tissue Distribution by Quantitative PCR

Analysis of genome integration of proviral sequences delivered by pseudotyped or VSV-G-deficient lentiviral vector particles encapsulated in OM-PBAEs was carried out generally as described in Examples 2 and 3 except that qPCR was performed on genomic DNA extracted from blood and tissues collected 7 days after the last intravenous injection or infusion of the products.

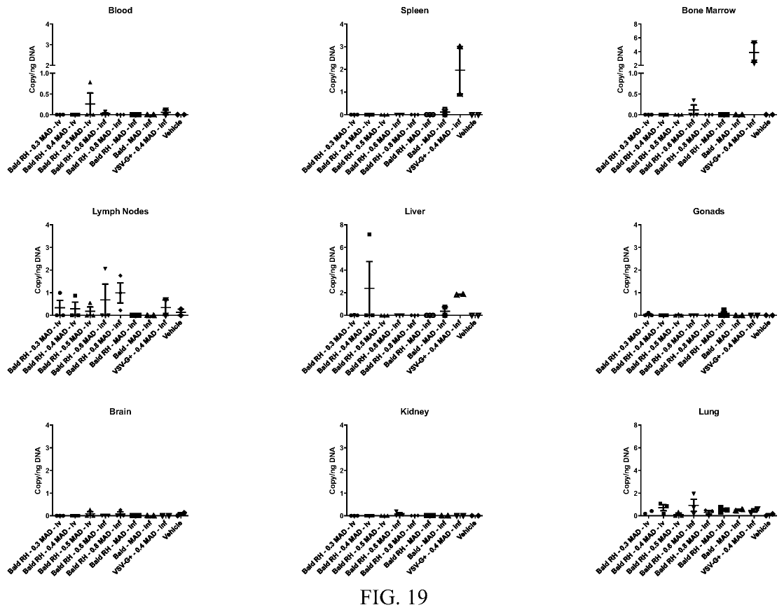

As previously described, qPCR analysis of biodistribution of pseudotyped lentiviral vectors revealed an integration of proviral sequences in spleen, liver, bone marrow collected on mice seven days after the last intravenous infusion as depicted in FIG. 19. VSV-G-deficient lentiviral vectors did not integrate in any of the collected organs. VSV-G-deficient lentiviral vectors encapsulated in DMSO-free OM-PBAE polymers showed a different biodistribution profile with unexpected subtle changes compared to results obtained with polymers formulated in DMSO. Here the integration profile varied with the injected dose. Integration in blood cells occurred with 3 intravenous injections (0.5 MAD) when a single infusion (0.6 MAD) was sufficient to transduce bone marrow cells. A dose-dependent integration of lentiviral vector was observed in the lymph nodes. More surprisingly integrations were detected in the lungs with two intravenous injections (0.4 MAD) when a single infusion (0.6 MAD). Given that lungs are a highly vascularized organ, signals may just come from transduced blood cells present in these tissues. Therefore, if the transduction of blood cells could be repeated with a dose equivalent to the positive condition found in example 3 (2×10¹¹ lentiviral particles), the formulation of OM-PBAE and the administration route seems to have a major influence on the fate of nanoparticles in the body.

Evaluation of Cellular GFP Expression by Flow Cytometry

Fresh and heparinized whole blood samples were collected from Balb/c mice 9 days before treatment (submandibular sampling) or 3 days and 7 days after the last intravenous injection or infusion of the lentiviral vector particles by cardiac puncture on animals anesthetized with 2% isoflurane. Moreover, spleen and bone marrow were collected at sacrifice. Single cell suspensions were obtained by meshing the tissues through a 100 μm cell strainer followed by a red blood cells lysis step according to protocol for dissociation of lymphoid tissues provided with RBC lysis buffer (Invitrogen Inc.).

The percentage of blood circulating cells expressing GFP was determined by flow cytometry with antibody panels previously described for leukocyte counts and recording the GFP fluorescence with the BL1 channel.

As shown in FIG. 20, GFP-positive eosinophils and monocytes/macrophages were found in blood and bone marrow across all treatments, reflecting background uptake by phagocytosis. However, VSV-G-deficient lentiviral vectors encapsulated in DMSO-free OM-PBAE polymers induced a dose-dependent expression of the GFP reporter in B lymphocytes, T lymphocytes and NK cells when administered via intravenous injections when bald and pseudotyped lentiviral vectors were not active on these cell types. Low GFP-positive cells were detected in spleens collected from mice treated with bald and pseudotyped lentiviral vectors. An increased rate of GFP-positive T lymphocytes, NK cells and eosinophils in the spleen and NK cells, B and T lymphocytes in the bone marrow occurred after all treatments with VSV-G-deficient lentiviral vectors encapsulated in DMSO-free OM-PBAE polymers.

Detailed analysis of the T lymphocyte population confirms that VSV-G-deficient lentiviral vectors encapsulated in DMSO-free OM-PBAE polymers induced a dose-dependent transduction of CD4+ and CD8+ sub-populations in the blood with both administration routes. Transduction of CD4+ and CD8+ cells occurred in the spleen as well but two and three intravenous injections were the most efficient treatments. For both organs, GFP expression levels were superior to those detected in mice injected with bald and pseudotyped lentiviral vectors. No significant difference was visible at the level of bone marrow.

Based on these results, VSV-G-deficient lentiviral vectors encapsulated in DMS-free OM-PBAE polymers can be safely injected systemically in a repeated manner and can efficiently deliver in vivo a transgene to leukocytes present in the blood but also in primary (bone marrow) and secondary (spleen) lymphoid organs without the need of any targeting agent or prior activation of proliferation that is normally required with lentiviral-mediated gene transfer.

What is claimed is:

1. A method of in vivo transduction of cells of a subject and expression of a transgene, the method comprising:
(a) providing a lentiviral vector nanoparticle comprising
(i) a lentiviral vector lacking a viral fusion protein and encoding the transgene; and
(ii) a plurality of oligopeptide modified poly(beta amino ester) (OM-PBAE) molecules forming a shell surrounding the lentiviral vector; and
(b) administering the lentiviral vector nanoparticle parenterally to the subject, whereby cells of the subject are transduced by the lentiviral vector and the transgene is expressed in the cells;
wherein the method has an improved safety profile compared to a method comprising administering a lentiviral vector comprising a viral fusion protein.

2. The method of claim 1, wherein said improved safety profile comprises one or more of lack of activation of immune cells, lack of change in body weight, lack of change in a blood cell count, lack of induction of a cytokine, and lack of hepatotoxicity.

3. The method of claim 2, wherein the safety profile comprises lack of induction of one or more cytokines selected from the group consisting of IL-2, IL-4, IL-5, TNF-a, and IFN-g.

4. The method of any of the preceding claims, wherein the lentiviral vector nanoparticle shows tropism for leucocytes without the use of a targeting moiety directed to a leucocyte-specific target.

5. The method of any of the preceding claims, wherein the lentiviral vector does not show tropism toward spleen, bone marrow, or liver.

6. The method of any of the preceding claims, wherein T cells of the subject are transduced.

7. The method of claim 6, wherein transduction and expression of the transgene do not require activation of the T cells.

8. The method of claim 6 or claim 7, wherein the transgene encodes a chimeric antigen receptor (CAR).

9. The method of claim 8, wherein the CAR has specificity for CD19.

10. The method of claim 8 or claim 9, wherein the expressed CAR is capable of directing killing by the T cell of a cell targeted by the CAR.

11. The method of any of the preceding claims, wherein the lentiviral vector nanoparticle further comprises a targeting moiety that directs the nanoparticle to a target cell.

12. The method of claim 10, wherein the targeting moiety has specificity for CD3.

13. The method of any of the preceding claims wherein the parenteral administration is by one or more intravenous injections or one or more intravenous infusions.

14. The method of any of the preceding claims, wherein the OM-PBAE is synthesized by a DMSO-free method.

15. The method of any of the preceding claims, wherein the OM-PBAE molecules comprise oligopeptides at both ends of the molecules, and wherein the end-modifying oligopeptides are the same or different at the two ends of each PBAE molecule.

16. The method of claim 15, wherein the oligopeptides comprise a sequence selected from the group consisting of RRR, KKK, HHH, DDD, and EEE.

17. The method of any of the preceding claims, further comprising preparing the lentiviral vector nanoparticles within four hours prior to administering the lentiviral vector nanoparticles.

18. The method of any of the preceding claims, further comprising preparing the lentiviral vector nanoparticles by a method comprising mixing a solution comprising OM-PBAE molecules with a solution comprising lentiviral vectors, whereby the lentiviral vectors become coated with PBAE molecules to form the lentiviral vector nanoparticles.

19. The method of claim 18, wherein said mixing is performed using a microfluidic device.

20. A kit for preparing lentiviral vector nanoparticles, the kit comprising:
(i) a plurality of lentiviral vectors in a first container;
(ii) a plurality of OM-PBAE molecules in a second container;
(iii) a microfluidic device configured to perform the mixing of claim 19; and
(iv) instructions for performing the method of claim 19.
